(19) United States
(12) Patent Application Publication
(10) Pub. No.: US 2010/0331269 A1
(43) Pub. Date: Dec. 30, 2010

(54) TRITERPENES DERIVATIVES AND USES THEREOF AS ANTITUMOR AGENTS OR ANTI-INFLAMMATORY AGENTS

(76) Inventors: Andre Pichette, Chicoutimi (CA); Jean Legault, Chicoutimi (CA); Charles Gauthier, Chicoutimi (CA)

Correspondence Address: GOUDREAU GAGE DUBUC, 2000 MCGILL COLLEGE, SUITE 2200, MONTREAL, QC, H3A 3H3, CA

(21) Appl. No.: 12/817,641

(22) Filed: Jun. 17, 2010

Related U.S. Application Data
(62) Division of application No. 11/924,789, filed on Oct. 26, 2007, now abandoned.
(60) Provisional application No. 60/863,215, filed on Oct. 27, 2006.
(60) Provisional application No. 60/914,784, filed on Apr. 30, 2007.

(30) Foreign Application Priority Data
Apr. 27, 2007 (CA) 2,586,614

(51) Int. Cl. A61K 31/704; A61K 31/57; A61P 35/00; A61P 29/00; G01N 33/574

(52) U.S. Cl. 514/26; 514/169; 435/7.23

(57) ABSTRACT

A compound of formula (I):

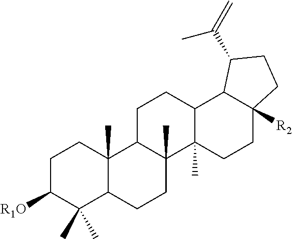

wherein
R₁ is selected from the group consisting of H, α-L-Rhamnopyranose, α-D-Mannopyranose, β-D-Xylopyranose, β-D-Glucopyranose, and α-D-Arabinopyranose; R₂ is selected from CH₃, COOH, CH₂OH, COOCH₃ and CH₂O-α-D-Arabinopyranose; with the proviso that the compound of formula (I) is not a compound of formula (I) wherein R₁ is β-D-Glucopyranose and R₂ is COOH; wherein R₁ is α-L-Rhamnopyranose and R₂ is CH₃; wherein R₁ is β-D-Glucopyranose and R₂ is CH₂OH; wherein R₁ is β-D-Xylopyranose and R₂ is CH₂OH; wherein R₁ is α-L-Rhamnopyranose and R₂ is COOCH₃, wherein R₁ is H and R₂ is CH₃; wherein R₁ is H and R₂ is CH₂OH; wherein R₁ is H and R₂ is COOH; or wherein R₁ is H and R₂ is COOCH₃,
or a pharmaceutically acceptable salt thereof.

### CROSS REFERENCE TO RELATED APPLICATIONS

This application claims priority on U.S. provisional application No. 60/863,215, filed on Oct. 27, 2006 and on 60/914,784 filed Apr. 30, 2007. All documents above are incorporated herein in their entirety by reference.

### FIELD OF THE INVENTION

The invention relates to triterpenes derivatives and uses thereof as antitumor agents or anti-inflammatory agents.

### BACKGROUND OF THE INVENTION

One-third of all individuals in the United States will develop cancer during their life. Although the five-year survival rate has risen dramatically as a result of progress in early diagnosis and therapy, cancer still remains second only to cardiac disease as a cause of death in the United States. Twenty percent of Americans die from cancer, half due to lung, breast, and colon-rectal cancer, and skin cancer remains a serious health hazard. Currently available therapies such as chemotherapy and radiotherapy are not effective against all types of cancer and have undesirable side effects (high toxicity). Therefore, there is a great need to develop effective antitumor agents having reduced side effects.

In the boreal forest of North America, pentacyclic triterpenes of the lupane-type such as lupeol, betulin and betulinic acid are found in the external bark of yellow (Betula alleghaniensis) and white (Betula papyrifera) birches. Betulinic acid is synthesized in a two-step process by taking advantage of the abundance of betulin in the bark of white birches. Betulinic acid has been shown to possess various medicinal properties including anti-inflammatory, anti-malarial and anti-HIV activities (Pato\check{c}ka, J., J. Appl. Biomed. 2003, 1, 7-12; Fujioka et al., J. Nat. Prod. 1994, 57, 243-247).

Antitumor data from various animal models utilizing betulinic acid have been extremely variable and apparently inconsistent. For example, betulinic acid was reported to demonstrate dose-dependent activity against the Walker 256 murine carcinosarcoma tumor system at dose levels of 300 and 500 mg/kg (milligrams per kilogram) body weight. In contrast, a subsequent report indicated the compound was inactive in the Walker 256 (400 mg/kg) and in the L1210 murine lymphocytic leukemia (200 mg/kg) models. Similarly, an antitumor activity of betulinic acid in the P-388 murine lymphocyte test system has been suggested. However, this activity was not confirmed by tests conducted by the National Cancer Institute. The anti-cancer activity of betulinic acid in neuroectodermal and melanoma tumour models has also been reported. Certain betulinic acid derivatives were also shown to possess anti-cancer activity using mouse sarcoma 180 cells implanted subcutaneously in nude mice. Betulinic acid 3-monoacetate, and betulinic acid methyl ester have been shown to exhibit ED50 values of 10.5 and 6.8 μg/ml, respectively, against P388 lymphocytic leukemia cells.

The present description refers to a number of documents, the content of which is herein incorporated by reference in their entirety.

### SUMMARY OF THE INVENTION

More specifically, in accordance with one aspect of the present invention, there is provided a compound of formula (I):

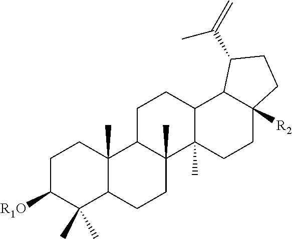

wherein R₁ is selected from the group consisting of H, α-L-Rhamnopyranose, α-D-Mannopyranose, β-D-Xylopyranose, β-D-Glucopyranose, and α-D-Arabinopyranose;

R₂ is selected from CH₃, COOH, CH₂OH, COOCH₃ and CH₂O-α-D-Arabinopyranose;

with the proviso that the compound of formula (I) is not a compound of formula (I) wherein R₁ is β-D-Glucopyranose and R₂ is COOH; wherein R₁ is α-L-Rhamnopyranose and R₂ is CH₃; wherein R₁ is β-D-Glucopyranose and R₂ is CH₂OH; wherein R₁ is β-D-Xylopyranose and R₂ is CH₂OH; wherein R₁ is α-L-Rhamnopyranose and R₂ is COOCH₃, wherein R₁ is H and R₂ is CH₃; wherein R₁ is H and R₂ is CH₂OH; wherein R₁ is H and R₂ is COOH; or wherein R₁ is H and R₂ is COOCH₃, or a pharmaceutically acceptable salt thereof.

In a specific embodiment of the compound, R₁ is β-D-Glucopyranose and R₂ is CH₃. In an other specific embodiment of the compound, R₁ is α-D-Arabinopyranose and R₂ is CH₃. In an other specific embodiment of the compound, R₁ is α-L-Rhamnopyranose and R₂ is CH₂OH. In an other specific embodiment of the compound, R₁ is α-D-Arabinopyranose and R₂ is CH₂OH. In an other specific embodiment of the compound, R₁ is α-D-Mannopyranose and R₂ is CH₂OH. In an other specific embodiment of the compound, R₁ is β-D-Glucopyranose and R₂ is COOCH₃. In an other specific embodiment of the compound, R₁ is α-D-Arabinopyranose and R₂ is COOCH₃. In an other specific embodiment of the compound, R₁ is α-L-Rhamnopyranose and R₂ is COOH. In an other specific embodiment of the compound, R₁ is α-D-Arabinopyranose and R₂ is COOH. In an other specific embodiment of the compound, R₁ is α-D-Mannopyranose and R₂ is COOH. In an other specific embodiment of the compound, R₁ is β-D-Xylopyranose and R₂ is COOH. In an other specific embodiment of the compound, R₁ is H and R₂ is CH₂O-α-D-Arabinopyranose.

In accordance with an other aspect of the present invention, there is provided a method of administering a compound of formula (I)

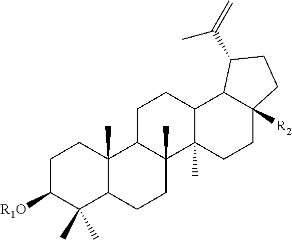

wherein R₁ is selected from the group consisting of hydrogen, acetate, α-L-Rhamnopyranose, α-D-Mannopyranose, β-D-Xylopyranose, β-D-Glucopyranose, and α-D-Arabinopyranose; R₂ is selected from CH₃, COOH, CH₂OH and COOCH₃; to a subject suffering from a cancer selected from the group consisting of melanoma, colorectal adenocarcinoma, lung carcinoma, liver carcinoma, breast adenocarcinoma, ovarian teratocarcinoma, prostate adenocarcinoma and glioma, with the proviso that the compound of formula (I) is not a compound of formula (I) wherein R₁ is hydrogen and R₂ is CH₃; wherein R₁ is hydrogen and R₂ is CH₂OH; wherein R₁ is hydrogen and R₂ is COOH; wherein R₁ is acetate and R₂ is CH₂OH; wherein R₁ is hydrogen and R₂ is COOCH₃, wherein R₁ is α-L-Rhamnopyranose and R₂ is CH₃; wherein R₁ is β-D-Glucopyranose and R₂ is CH₂OH; wherein R₁ is β-D-Xylopyranose and R₂ is CH₂OH; wherein R₁ is α-L-Rhamnopyranose and R₂ is COOCH₃, or wherein R₁ is β-D-Glucopyranose and R₂ is COOH.

In a specific embodiment of the method, R₁ is acetate and R₂ is COOH. In an other specific embodiment of the method, R₁ is β-D-Glucopyranose and R₂ is CH₃. In an other specific embodiment of the method, R₁ is α-D-Arabinopyranose and R₂ is CH₃. In an other specific embodiment of the method, R₁ is α-L-Rhamnopyranose and R₂ is CH₂OH. In an other specific embodiment of the method, R₁ is α-D-Arabinopyranose and R₂ is CH₂OH. In an other specific embodiment of the method, R₁ is α-D-Mannopyranose and R₂ is CH₂OH. In an other specific embodiment of the method, R₁ is β-D-Glucopyranose and R₂ is COOCH₃. In an other specific embodiment of the method, R₁ is α-D-Arabinopyranose and R₂ is COOCH₃. In an other specific embodiment of the method, R₁ is α-L-Rhamnopyranose and R₂ is COOH. In an other specific embodiment of the method, R₁ is α-D-Arabinopyranose and R₂ is COOH. In an other specific embodiment of the method, R₁ is α-D-Mannopyranose and R₂ is COOH. In an other specific embodiment of the method, R₁ is β-D-Xylopyranose and R₂ is COOH.

In accordance with an other aspect of the present invention, there is provided a method of administering methyl betulinate to a subject suffering from colorectal adenocarcinoma or lung carcinoma.

In accordance with an other aspect of the present invention, there is provided a method of administering 3-β-D-glucopyranose betulinic acid to a subject suffering from colorectal adenocarcinoma or lung carcinoma.

In a specific embodiment of the methods of the present invention, the administration is parenteral or systemic. In an other specific embodiment of the methods, the administration is at a tumour site. In an other more specific embodiment of the method, the cancer is lung carcinoma. In an other more specific embodiment of the method, the administration is in a dosage of about 0.5 mg/kg to about 50 mg/kg. In an other more specific embodiment of the method, the administration is in a dosage of about 4 mg/kg to about 40 mg/kg.

In accordance with an other aspect of the present invention, there is provided a compound of formula (II):

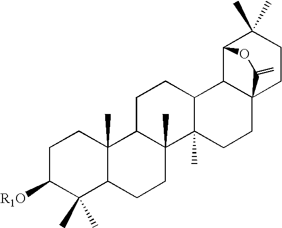

wherein R1 is selected from β-D-Glucopyranose and β-D-Galactopyranose, and a pharmaceutically acceptable salt thereof.

In a specific embodiment of the compound, R1 is β-D-Glucopyranose. In an other specific embodiment of the compound, R1 is β-D-Galactopyranose.

In accordance with an other aspect of the present invention, there is provided a method of administering a compound of the present invention to a subject suffering from a cancer selected from the group consisting of, colorectal adenocarcinoma, lung carcinoma, liver carcinoma, breast adenocarcinoma, ovarian teratocarcinoma, prostate adenocarcinoma and glioma.

In accordance with an other aspect of the present invention, there is provided a pharmaceutical composition comprising the compound of the present invention and a pharmaceutically acceptable diluent, carrier or excipient.

In a specific embodiment of the pharmaceutical composition, the compound is in a racemate form.

In accordance with an other aspect of the present invention, there is provided a method of identifying a tumor amenable to treatment with the compound of the present invention, comprising contacting a sample of cells isolated from said tumor with the compound, wherein an IC₅₀ of the compound against the sample of cells that is smaller than or equal to 50 μM in is indicative that the tumor is amenable to treatment with said compound.

In a specific embodiment of the method, said sample of cells is from a biopsy sample from a subject. In an other specific embodiment of the method, said sample of cells is from a biological fluid obtained from a subject.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents the chemical structure of lupeol, betulin and betulinic acid;

FIG. 2 presents the synthesis and structure of triterpenes and derivatives (1, 2, 4-6, 9-17, 25-27, 31, 33-38). Reagents and conditions: (a) Ac₂O, Py, DMAP, 0° C.-room temperature (rt), 5 h; (b) Mg(OCH₃)₂, CH₃OH-THF, room temperature, 4 h; (c) Ac₂O, CH₂Cl₂, room temperature, 24 h; (d) (i) Trichloroacetimidate, TMSOTf, 4 Å MS, CH₂Cl₂, room temperature, 30 min.; (ii) CH₃OH-THF-H₂O 1:2:1, NaOH 0.25 N, room temperature, 3-24 h; (e) CH₃OH-THF—H₂O 1:2:1, NaOH 0.25 N, room temperature, 2 h;

FIG. 3 presents the synthesis and structure of other triterpenes and derivatives (3, 7-8, 18-24, 28-30, 32, 39-44). Reagents and conditions: (a) DBU, CH₃I, THF, 0° C.-room temperature, 24 h; (b) (i) Trichloroacetimidate, TMSOTf, 4 Å MS, CH₂Cl₂, room temperature, 30 min.; (ii) CH₃OH-THF—H₂O 1:2:1, NaOH 0.25 N, room temperature, 3 h; (c) AIIBr, K₂CO₃, 55° C., 7 h; (d) Pd⁰(PPh₃)₄, PPh₃, pyrrolidine, THF, 24 h; (e) Ac₂O, CH₂Cl₂, room temperature, 24 h; (f) (i) FeCl₃/SiO₂, CH₂Cl₂, reflux, 3 h; (ii) CH₃OH-THF—H₂O 1:2:1, NaOH 0.25 N, room temperature, 2 h;

FIG. 4 presents the structure of the sugars used for the synthesis of glycosides;

FIG. 5 presents the predicted absorption, distribution, metabolism and excretion of different triterpenes and triterpene derivatives of the present invention;

FIG. 6 presents results of in vivo antitumoral activity of betulinic acid (BetA) and 3-O-α-L-rhamnopyranoside betulinic acid (RhaBetA) against Lewis lung cancer-bearing mice (tumours measured on day 11-13); and

FIG. 7 presents the effect of RhaBetA and BetA treatments on the weight of mice on day 13.

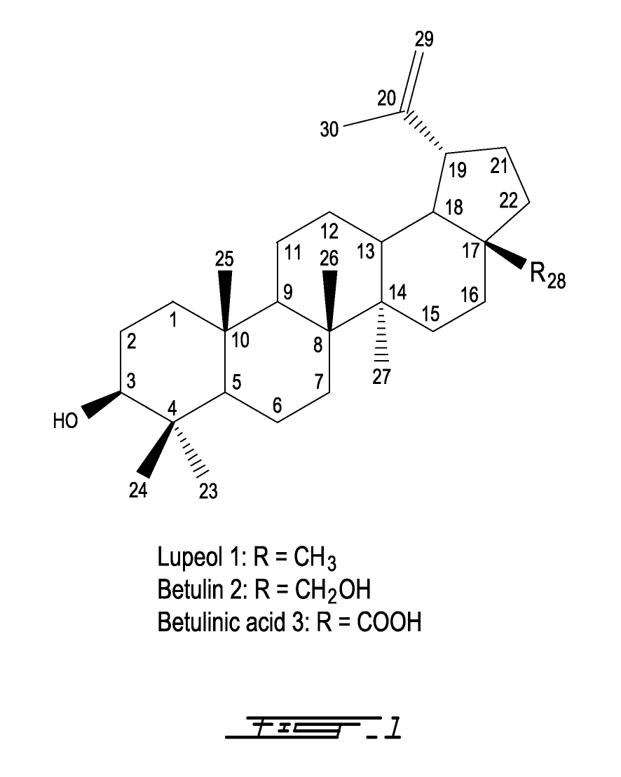

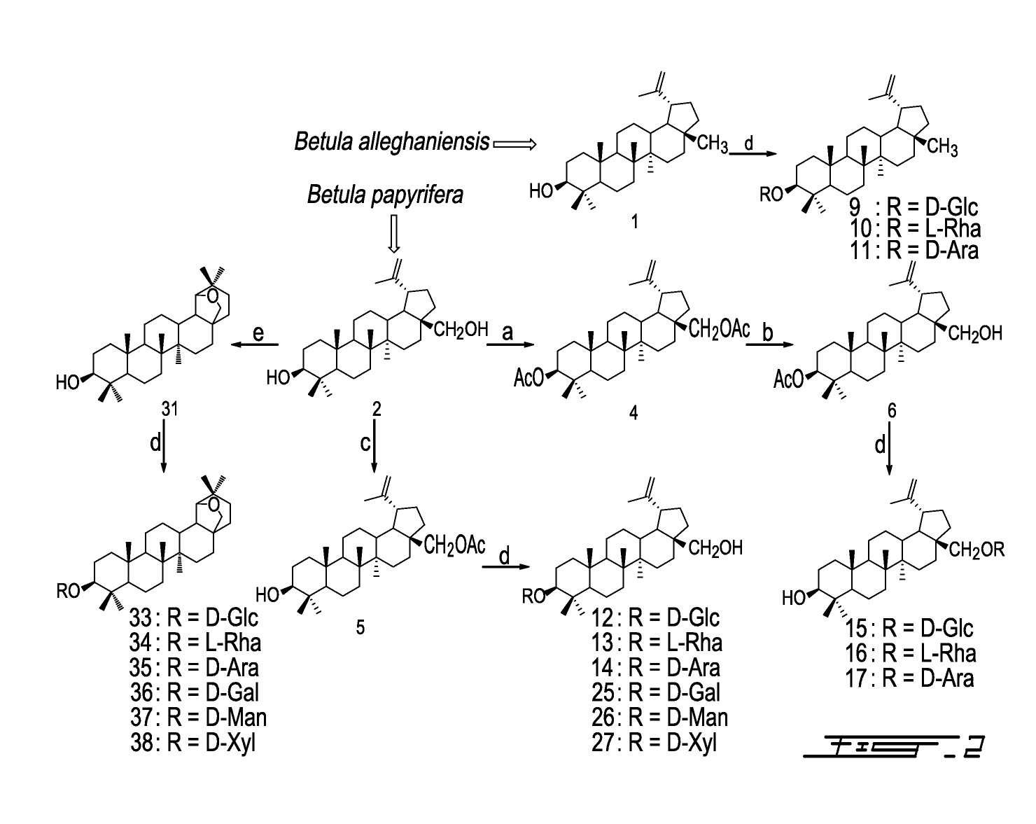

### DESCRIPTION OF ILLUSTRATIVE EMBODIMENTS

The term “pharmaceutically acceptable salts” as used herein refers herein to, without being so limited, salts derived from the carboxyl groups of the compound of the invention (partial structure thereof: —COOX; X represents an arbitrarily selected cationic substance) and in the present invention, these salts are not restricted to specific ones inasmuch as they are currently used in foods and beverages and medical or pharmaceutical compositions. Specific examples thereof include alkali metal salts such as sodium, potassium and lithium salts; alkaline earth metal salts such as calcium, magnesium, barium and zinc salts; alkylamine salts such as salts with, for instance, ammonia, methylamine, dimethylamine, trimethylamine, ethylamine, diethylamine, triethylamine, propylamine, butylamine, tetrabutylamine, pentylamine and hexylamine; alkanolamine salts such as salts with, for instance, ethanolamine, diethanolamine, triethanolamine, propanolamine, dipropanolamine, isopropanolamine and diisopropanolamine; salts with other organic amines such as piperazine and piperidine; and salts with basic amino acids such as lysine, arginine, histidine and tryptophan. On the whole, these salts have solubility in water higher than that of the original compounds and therefore, the salts are preferably used, in particular, in aqueous systems in the present invention.

As used herein the term “compound of formula I” is meant to include D-enantiomers, L-enantiomers and racemates of the compound of formula I.

The term “subject” or “patient” as used herein refers to an animal, preferably a mammal, and most preferably a human who is the object of treatment, observation or experiment. A “therapeutically effective amount” refers to an amount effective, at dosages and for periods of time necessary, to achieve the desired therapeutic result, such as a reduction of tumour growth and in turn a reduction in cancer-related disease progression. A therapeutically effective amount of the above-mentioned compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

The term “treating cancer” or “treatment of cancer” as used herein includes at least one of the following features: alleviation of the symptoms associated with the cancer, a reduction in the extent of the cancer (e.g. a reduction in tumor growth), a stabilization of the state of the cancer (e.g. an inhibition of tumor growth), a prevention of further spread of the cancer (e.g. a metastasis), a prevention of the occurrence or recurrence of a cancer, a delaying or retardation of the progression of the cancer (e.g. a reduction in tumor growth) or an improvement in the state of the cancer (e.g. a reduction in tumor size).

The compounds of the present invention can be orally or parenterally and stably administered to human and animals to act as, for instance, a drug or a quasi-drug. In this respect, examples of parenteral administration include intravenous injection, intra-arterial injection, intramuscular injection, subcutaneous injection, intracutaneous injection, intraperitoneal injection, intra-spinal injection, peridural injection, percutaneous administration, perpulmonary administration, pernasal administration, perintestinal administration, administration through oral cavity and permucosal administration and examples of dosage forms used in such parenteral administration routes include injections, suppositories (such as rectal suppositories, urethral suppositories and vaginal suppositories), liquids for external use (such as injections, gargles, mouth washes, fomentations, inhalants, sprays, aerosols, enema, paints, cleaning agents, disinfectants, nasal drops and ear drops), cataplasms, percutaneous absorption tapes, external preparations for the skin, ointments (such as pastes, liniments and lotions). In addition, examples of pharmaceutical preparations for oral administration include tablets for internal use (such as uncoated tablets, sugar-coated tablets, coating tablets, enteric coated tablets and chewable tablets), tablets administered to oral cavity (such as buccal preparations, sublingual tablets, troches and adhesive tablets), powders, capsules (such as hard capsules and soft capsules), granules (such as coated granules, pills, troches, liquids preparations or pharmaceutically acceptable sustained release pharmaceutical preparations). Specific examples of liquid preparations capable of being orally administered are solutions for internal use, shake mixtures, suspensions, emulsions, syrups, dry syrups, elixirs, infusion and decoction and lemonades.

The invention also relates to a pharmaceutical composition comprising the above-mentioned compound and a pharmaceutically acceptable diluent, carrier or excipient. As used herein “pharmaceutically acceptable carrier” or “diluent” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound, use thereof in the pharmaceutical compositions of the invention is contemplated. Supplementary active compounds can also be incorporated into the compositions.

Pharmaceutical composition within the scope of the present invention desirably contain the active agent (the above-mentioned compound) in an amount effective to achieve the desired therapeutic effect while avoiding adverse side effects. Pharmaceutically acceptable preparations and salts of the active agent are within the scope of the present invention and are well known in the art. The amount of the therapeutic or pharmaceutical composition which is effective in the treatment of a particular disease, disorder or condition will depend on the nature and severity of the disease, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 100 mg/kg/day will be administered to the subject. Effective doses may be extrapolated from dose response curves derived from in vitro or animal model test systems. For example, in order to obtain an effective mg/kg dose for humans based on data generated from mice studies, the effective mg/kg dosage in rat is divided by 12.3.

The pharmaceutical compositions of the present invention can be delivered in a controlled release system. For example, polymeric materials can be used (see Smolen and Ball, Controlled Drug Bioavailability, Drug product design and performance, 1984, John Wiley & Sons; Ranade and Hollinger, Drug Delivery Systems, pharmacology and toxicology series, 2003, 2nd edition, CRRC Press), or a pump may be used (Saudek et al., 1989, N. Engl. J. Med. 321:574).

Compounds of the present invention may also be delivered by the use of monoclonal antibodies as individual carriers to which the compound molecules are coupled. The compounds of the present invention may also be coupled to a class of biodegradable polymers useful in achieving controlled release of the drug, for example, polylactic acid, polyorthoesters, cross-linked amphipathic block copolymers and hydrogels, polyhydroxy butyric acid and polydihydropyrans.

In a further aspect, the present invention provides a method of preventing or inhibiting tumour growth comprising contacting said cell with a therapeutically effective amount of the above-mentioned compound. The tumours to which the compound of the present invention can be applied include swellings and true tumors including benign and malignant tumors. Specific examples of such tumors are gliomas such as astrocytoma, glioblastoma, medulloblastoma, oligodendroglioma, ependymoma and choroid plexus papilloma; cerebral tumors such as meningioma, pituitary adenoma, neurioma, congenital tumor, metastatic cerebral tumor; squamous cell carcinoma, lymphoma, a variety of adenomas and pharyngeal cancers resulted from these adenomas such as epipharyngeal cancer, mesopharyngeal cancer and hypopharyngeal cancer; laryngeal cancer, thymoma; mesothelioma such as pleural mesothelioma, peritoneal mesothelioma and pericardial mesothelioma; breast cancers such as thoracic duct cancer, lobular carcinoma and papillary cancer; lung cancers such as small cell carcinoma, adenocarcinoma, squamous cell carcinoma, large cell carcinoma and adenosquamous carcinoma; gastric carcinoma; esophageal carcinomas such as cervical esophageal carcinomas, thoracic esophageal carcinomas and abdominal esophageal carcinomas; carcinomas of large intestine such as rectal carcinoma, S-like (sigmoidal) colon carcinoma, ascending colon carcinoma, lateral colon carcinoma, cecum carcinoma and descending colon carcinoma; hepatomas such as hepatocellular carcinoma, intrahepatic hepatic duct carcinoma, hepatocellular blastoma and hepatic duct cystadenocarcinoma; pancreatic carcinoma; pancreatic hormone-dependent tumors such as insulinoma, gastrinoma, VIP-producing adenoma, extrahepatic hepatic duct carcinoma, hepatic capsular carcinoma, perial carcinoma, renal pelvic and uretal carcinoma; urethral carcinoma; renal cancers such as renal cell carcinoma (Grawitz tumor), Wilms' tumor (nephroblastoma) and renal angiomyolipoma; testicular cancers or germ cell tumors such as seminoma, embryonal carcinoma, vitellicle tumor, choriocarcinoma and teratoma; prostatic cancer, bladder cancer, carcinoma of vulva; hysterocarcinomas such as carcinoma of uterine cervix, uterine corpus cancer and solenoma; hysteromyoma, uterine sarcoma, villous diseases, carcinoma of vagina; ovarian germ cell tumors such as dysgerminoma, vitellicle tumor, premature teratoma, dermoidal cancer and ovarian tumors such as ovarian cancer; melanomas such as nevocyte and melanoma; skin lymphomas such as mycosis fungoides, skin cancers such as endoepidermal cancers resulted from skin cancers, prodrome or the like and spinocellular cancer, soft tissue sarcomas such as fibrous histiocytomatosis, liposarcoma, rhabdomyosarcoma, leiomyosarcoma, synovial sarcoma, sarcoma fibroplasticum (fibrosarcoma), neurioma, hemangiosarcoma, fibrosarcoma, neurofibrosarcoma, perithelioma (hemangiopericytoma) and alveolar soft part sarcoma, lymphomas such as Hodgkin lymphoma and non-Hodgkin lymphoma, myeloma, plasmacytoma, acute myelocytic (myeloid) leukemia and chronic myeloid leukemia, leukemia such as adult T-cell leukemic lymphoma and chronic lymphocytic leukemia, chronic myeloproliferative diseases such as true plethora, essential thrombocythemia and idiopathic myelofibrosis, lymph node enlargement (or swelling), tumor of pleural effusion, ascitic tumor, other various kinds of adenomas, lipoma, fibroma, hemangeoma, myoma, fibromyoma and endothelioma.

The terms “biological sample” are meant to include any tissue or material derived from a living or dead (human) that may contain tumour cells. Samples include, without being so limited, any tissue or material such as blood or fraction thereof, tissue biopsies (lung, prostate, kidney, skin, stomach, intestine, liver, lymph nodes, pancreas, breast, etc.), bronchial aspiration, sputum, saliva or urine from test patients (suspected cancer patients and control patients) or other biological fluids or tissues.

By the term “normal cell” (control sample) is meant herein a cell sample that does not contain a specifically chosen cancer. Control samples can be obtained from patients/individuals not afflicted with cancer. Alternatively, a control sample can be taken from a non-afflicted tissue of a suspected cancer patient. Other types of control samples may also be used, such as a non-tumour cell line.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### Example 1

### Materials and Methods

Chemicals

Air and water sensitive reactions were performed in flame-dried glassware under a nitrogen or argon atmosphere. Moisture sensitive reagents were introduced via a dry syringe. Dichloromethane was distilled from CaH₂. THF was distilled from sodium with benzophenone as indicator of moisture. Betulinic acid (3) was purchased from Indofine Chemical Company. Tetrakistriphenylphosphine palladium(0) was prepared as mentioned in the literature (Coulson, D. R. Inorg. Syn. 1972, 13, 121-124) and stored under nitrogen. All other chemicals and materials were purchased from Sigma-Aldrich and were used as received. Flash chromatography was carried out using 60-230 mesh silica gel. Analytical thin-layer chromatography was performed with silica gel 60 F₂₅₄, 0.25 mm pre-coated TLC plates and visualized using UV₂₅₄ and cerium molybdate (2 g Ce(SO₄)₄(NH₄)₄, 5 g MoO₄(NH₄)₂, 200 mL H₂O, 20 mL H₂SO₄) with charring. All of the chemical yields are not optimized and generally represent the result of the mean of two experiments. ¹H NMR spectra were recorded at 400 MHz and ¹³C NMR were recorded at 100 MHz on an Avance 400 Bruker spectrometer equipped with a 5 mm QNP probe. Elucidations of chemical structures were based on ¹H, ¹³C, DEPT135, COSY, HSQC and HMBC NMR experiments. Chemical shifts are reported in parts per million (ppm) relative to residual solvent peaks. Signals are reported as m (multiplet), s (singlet), d (doublet), t (triplet), q (quinquet), c (complex), brs (broad singlet) and coupling constants are reported in hertz (Hz). Melting points were determined in capillaries and are uncorrected. Optical rotations were obtained using sodium D line at ambient temperature on a Jasco DIP-360 digital polarimeter. Mass spectral data (HRMS) were obtained at the Department of Chemistry, Queen's University, Ontario, Canada.

Isolation of Lupeol (Compound 1)

The finely ground external bark (150 g) of the yellow birch (Betula alleghaniensis Britton), collected in Saguenay, Quebec, Canada, was extracted in CHCl₃ (1 L) with a soxhlet apparatus, refluxed for 1 day and purified by flash chromatography (CH₂Cl₂ to CH₂Cl₂:CH₃OH 99:1) to give 1 as a white powder (1.77 g; 1.2%): R_{f} 0.63 (CH₂Cl₂); mp 213-215° C., lit.⁴⁹ mp 215-216° C.; [α]²⁰_{D} +19.6° (c 1.2, CHCl₃), lit.⁴⁹ [α]_{D} +26.4° (CHCl₃). ¹H and ¹³C NMR spectral data of 1 were in agreement with those published in the literature (Setzer, W. N. et al., Min. Rev. Med. Chem. 2003, 3, 540-556): HR-EI-MS m/z 426.3854 [M]⁺ (calculated for C₃₀H₅₀O, 426.3862).

Isolation of Betulin (Compound 2)

The finely ground external bark (150 g) of the white birch (Betula papyrifera Marsh), collected in Saguenay, Quebec, Canada, was soaked in CH₂Cl₂ (1 L), refluxed for 1 day and purified by flash chromatography (CH₂Cl₂ to CH₂Cl₂:CH₃OH 49:1) to give 2 as a white powder (25 g, 17%): R_{f} 0.17 (CH₂Cl₂); mp 250-252° C., (Connolly, J. D.; Hill, R. A. In Dictionary of Triterpenoids. Di- and higher terpenoids; Chapman & Hall: Cambridge, 1991; Vol. 2, 1460 p.) mp 251-252° C.; [α]²⁰_{D} +19.1° (c 0.67, C₅H₅N), (Connolly, J. D., supra) [α]¹⁵_{D} +20.0° (C₅H₅N). ¹H and ¹³C NMR spectral data of 2 were in agreement with those published in the literature (Tinto, W. F.; Blair, L. C.; Alli, A. J. Nat. Prod. 1992, 55, 395-398): HR-EI-MS m/z 442.3804 [M]⁺ (calculated for C₃₀H₅₀O₂, 442.3811).

3,28-Diacetoxybetulin (Compound 4)

Acetic anhydride (4.8 mL, 50 mmol) was added to a cooled solution (ice-water bath) of 2 (7.50 g, 17 mmol) in pyridine (182 mL) with DMAP (100 mg, 0.82 mmol) as catalyst. After stirring at room temperature for 5 h, the mixture was diluted with CH₂Cl₂, then, washed with cold H₂SO₄ 3 N, saturated NaHCO₃ solution and brine. The solvents of the dried solution (MgSO₄) were evaporated under reduced pressure and the residue was purified by flash chromatography (Hexanes to Hexanes:EtOAc 97:3) to give 4 as a white crystalline powder (8.48 g, 95%): R_{f} 0.74 (CH₂Cl₂); mp 216-218° C., (Connolly, J. D., supra) mp 223-224° C.; [α]²⁰_{D} +19.7° (c 1.67, CHCl₃), (Connolly, J. D., supra) [α]²⁰_{D} +22°. ¹H and ¹³C NMR spectral data of 4 were in agreement with those published in the literature (Hiroya, K. et al., Bioorg. Med. Chem. 2002, 10, 3229-3236): HR-ESI-MS m/z 549.3925 [M+Na]⁺ (calculated for C₃₄H₅₄O₄Na, 549.3920).

28-Acetoxybetulin (Compound 5)

Acetic anhydride (300 mL, 3.1 mol) was added to a solution of 2 (11.6 g, 26.2 mmol) in CH₂Cl₂ (750 mL). After stirring overnight at room temperature, the mixture was washed exhaustively with saturated NaHCO₃ solution and brine. The solvents of the dried solution (MgSO₄) were evaporated under reduced pressure and the residue was purified by flash chromatography (CH₂Cl₂ to CH₂Cl₂:CH₃OH 49:1) to give 5 as a white powder (9.28 g, 73%): R_{f} 0.31 (CH₂Cl₂); mp 210-212° C.; [α]²⁰_{D} +8.5° (c 1.58, CHCl₃). ¹H and ¹³C NMR spectral data of 5 were in agreement with those published in the literature (Hiroya, K., supra; Ohara, S.; Hishiyama, S. Mokuzai Gakkaishi 1994, 40, 444-451): HR-EI-MS m/z 484.3903 [M]⁺ (calculated for C₃₂H₅₂O₃, 484.3916).

3-Acetoxybetulin (Compound 6)

A solution of Mg(OCH₃)₂ in CH₃OH (224 mL, 8%) was added under N₂ to a solution of 4 (6.14 g, 11.7 mmol) in dry THF (181 mL) and dry CH₃OH (542 mL). After stirring 4 h at room temperature, the mixture was acidified with HCl 10% and extracted with CH₂Cl₂ (3×). Then, the organic layer was washed with saturated NaHCO₃ solution and brine. The solvents of the dried solution (MgSO₄) were evaporated under reduced pressure and the residue was purified by flash chromatography (Hexanes to Hexanes:EtOAc 9:1) to give 6 as a white solid (4.80 g, 85%): R_{f} 0.49 (CH₂Cl₂); mp 258-260° C., (Xu, Y.-C. et al., J. Org. Chem. 1996, 61, 9086-9089) mp 256-258° C.; [α]²⁰_{D} +25.7° (c 0.92, CHCl₃). ¹H and ¹³C NMR spectral data of 6 were in agreement with those published in the literature (Xu, Y.-C., supra): HR-EI-MS m/z 484.3904 [M]⁺ (calculated for C₃₂H₅₂O₃, 484.3916).

Methyl Betulinate (Compound 7)

DBU (0.17 mL, 1.1 mmol) and CH₃I (0.21 mL, 3.3 mmol) were slowly added under N₂ to a cooled solution (ice-water bath) of 3 (502 mg, 1.09 mmol) in dry THF (10 mL). The reaction was stirred overnight at room temperature, then filtered off and washed with dry THF. The filtrate and the combined washings were concentrated to give a yellow solid. This residue was acidified (HCl 6N) and extracted with CH₂Cl₂ (3×). After that, the organic layer was washed with H₂O, dried (MgSO₄) and then the solvents were evaporated under reduced pressure. The resulting residue was purified by flash chromatography (CH₂Cl₂) to give 7 as a white powder (367 mg, 71%): R_{f} 0.54 (CH₂Cl₂); mp 218-220° C., (Ziegler, H. L. et al., Bioorg. Med. Chem. 2004, 12, 119-127) 217-220° C.; [α]²⁰_{D} +1.3° (c 0.58, CHCl₃), (Ziegler, H. L., supra) [α]²⁵_{D} +5° (c 0.17, CHCl₃), (Kojima, H. et al., Phytochemistry 1987, 26, 1107-1111) [α]²⁶_{D} +4.0° (c 0.5, CHCl₃). ¹H and ¹³C NMR spectral data of 7 were in agreement with those published in the literature (Kojima, H., supra; Takeoka, G. et al., J. Agr. Food Chem. 2000, 48, 3437-3439; Yagi, A. et al., Chem. Pharm. Bull. 1978, 26, 1798-1802): HR-EI-MS m/z 470.3744 [M]⁺ (calculated for C₃₁H₅₀O₃, 470.3760).

Allyl betulinate (Compound 8)

Allyl bromide (0.19 mL, 2.2 mmol) and K₂CO₃ (454 mg, 3.28 mmol) were added to a solution of 3 (501 mg, 1.10 mmol) in DMF (7 mL). The reaction mixture was stirred 7 h at 55° C. After cooling, EtOAc was added and the organic layer was washed with 1N HCl. The aqueous layer was extracted with EtOAc (3×) and the combined organic layers were washed with saturated NaHCO₃ and brine. After the solution was dried (MgSO₄), the solvents were evaporated under reduced pressure. The resulting residue was purified by flash chromatography (CH₂Cl₂) to give 8 as a white crystalline powder (458 mg, 84%): R_{f} 0.58 (CH₂Cl₂:CH₃OH 99:1); mp 152-154° C.; [α]²⁰_{D} +3.9° (c 1.00, CHCl₃). ¹H NMR (CDCl₃) δ: 0.77, 0.83, 0.92 (all s, each 3H, H-24, H-25, H-26), 0.97 (s, 6H, H-23, H-27), 1.69 (s, 3H, H-30), 3.02 (m, 1H, H-19), 3.19 (dd, 1H, J=11.0 Hz, J=5.1 Hz, H-3), 4.58 (m, 2H, CH₂CH═CH₂), 4.61 (brs, 1H, H-29α), 4.74 (brs, 1H, H-29β), 5.24 (d, 1H, J=10.5 Hz, CH₂CH═CH₂, Ha), 5.35 (d, 1H, J=17.1 Hz, CH₂CH═CH₂, Hβ), 5.94 (ddt, 1H, J=17, 1 Hz, J=10, 5 Hz, J=5.7 Hz, CH₂CH═CH₂), 0.69-2.28 (all m, remaining protons). ¹³C NMR (CDCl₃) δ: 14.75, 15.44, 16.00, 16.19, 18.33, 19.44, 20.92, 25.56, 27.43, 28.04, 29.68, 30.61, 32.15, 34.36, 37.03, 37.22, 38.24, 38.77, 38.89, 40.77, 42.42, 46.94, 49.48, 50.59, 55.39, 56.59, 64.61 (CH₂CH═CH₂), 78.91 (C-3), 109.64 (C-29), 118.15 (CH₂CH═CH₂), 132.56 (CH₂CH═CH₂), 150.53 (C-20), 175.72 (C-28). HR-ESI-MS m/z 497.3985 [M+Hr]⁺ (calculated for C₃₃H₅₃O₃, 497.3995).

3-O-6-D-Glucopyranoside of lupeol (Compound 9)

The acceptor 1 (1.01 g, 2.34 mmol), and the donor 47 (2.60 g, 3.52 mmol) were stirred in dry CH₂Cl₂ (80 mL) for 1 h with 4 Å MS. At this time, TMSOTf (24 μL, 0.13 mmol) was added under Ar while keeping rigorous anhydrous conditions. The reaction was usually performed in 30 min, then quenched by addition of Et₃N (0.3 mL). The solvents were evaporated under reduced pressure and the resulting residue was immediately dissolved in a NaOH 0.25 N solution of CH₃OH:THF:H₂O 1:2:1 (240 mL). The reaction was stirred at room temperature for 2 h, dissolved in CH₂Cl₂ and washed with HCl 10% and brine. Once the solution was dried (MgSO₄), the solvents were evaporated under reduced pressure and the residue was purified by flash chromatography (CH₂Cl₂:CH₃OH, 49:1 to 47:3) to give 9 as a white powder (1.38 g, 90%, 2 steps): R_{f} 0.24 (CH₂Cl₂:CH₃OH 9:1); mp 176-178° C.; [α]²⁰_{D} +7.9° (c 0.50, CHCl₃). ¹H NMR (CDCl₃) δ: 0.79, 0.80, 0.83, 0.93, 0.99, 1.02 (all s, each 3H, H-23, H-24, H-25, H-26, H-27, H-28), 1.68 (s, 3H, H-30), 2.37 (m, 1H, H-19), 2.63 (brs, 4H, 4×OH), 3.13 (dd, 1H, J=11.2 Hz, J=4.8 Hz, H-3), 3.36 (m, 1H, H′-5), 3.42 (t, 1H, J=8.3 Hz, H′-2), 3.58 (q, 2H, J=8.7 Hz, H′-3, H′-4), 3.80 (dd, 1H, J=11.8 Hz, J=4.2 Hz, H′-6a), 3.86 (dd, 1H, J=12.0 Hz, J=3.1 Hz, H′-6β), 4.36 (d, 1H, J=7.7 Hz, H′-1), 4.57 (brs, 1H, H-29a), 4.69 (brs, 1H, H-29β), 0.67-1.92 (all m, remaining protons). ¹³C NMR (CDCl₃) δ: 14.70, 16.15, 16.38, 16.74, 18.16, 18.35, 19.50, 21.00, 25.26, 26.48, 27.60, 28.09, 30.02, 34.46, 35.74, 37.02, 38.20, 38.93, 39.35, 40.15, 40.99, 42.95, 43.17, 48.15, 48.45, 50.57, 55.77, 61.94 (C′-6), 69.69 (C′-4), 73.98 (C′-2), 75.29 (C′-5), 76.51 (C′-3), 90.29 (C-3), 105.32 (C′-1), 109.54 (C-29), 151.08 (C-20). HR-ESI-MS m/z 611.4267 [M+Na]⁺ (calculated for C₃₆H₆₀O₆Na, 611.4287).

3-O-α-L-Rhamnopyranoside of lupeol (Compound 10)

This compound was prepared from the acceptor 1 (502 mg, 1.18 mmol), and the donor 49 (1.09 g, 1.76 mmol) in the same manner as that described for compound 9. Purification by flash chromatography (CH₂Cl₂:CH₃OH, 49:1 to 47:3) afforded 10 as a white powder (485 mg, 72%, 2 steps): R_{f} 0.33 (CH₂Cl₂:CH₃OH 9:1); mp 214-216° C.; [α]²⁰_{D}-17.9° (c 0.50, CHCl₃). ¹H NMR (CDCl₃) δ: 0.75, 0.79, 0.83, 0.90, 0.94, 1.02 (all s, each 3H, H-23, H-24, H-25, H-26, H-27, H-28), 1.28 (d, 3H, J=6.1 Hz, H′-6), 1.69 (s, 3H, H-30), 2.38 (m, 1H, H-19), 3.07 (dd, 1H, J=11.3 Hz, J=4.8 Hz, H-3), 3.43 (t, 1H, J=9.2 Hz, H′-4), 3.77 (t, 1H, J=5.2 Hz, H′-3), 3.81 (dd, 1H, J=9.0 Hz, J=6.1 Hz, H′-5), 3.95 (brs, 1H, H′-2), 4.57 (brs, 1H, H-29a), 4.69 (brs, 1H, H-29β), 4.82 (brs, 1H, H′-1), 0.68-1.93 (all m, remaining protons). ¹³C NMR (CDCl₃) δ: 14.55, 15.98, 16.15, 16.25, 17.35 (C′-6), 18.01, 18.30, 19.33, 20.95, 25.14, 25.52, 27.44, 28.19, 29.86, 34.25, 35.59, 36.89, 38.05, 38.64, 39.06, 40.01, 40.85, 42.83, 43.02, 48.00, 48.31, 50.40, 55.45, 67.65 (C′-5), 71.26 (C′-2), 71.98 (C′-3), 74.00 (C′-4), 89.71 (C-3), 101.67 (C′-1), 109.33 (C-29), 151.01 (C-20). HR-ESI-MS m/z 595.4335 [M+Na]⁺ (calculated for C₃₆H₆₀O₅Na, 595.4338).

3-O-α-D-Arabinopyranoside of Lupeol (Compound 11)

This compound was prepared from the acceptor 1 (251 mg, 0.59 mmol), and the donor 51 (531 mg, 0.88 mmol) in the same manner as that described for compound 9. Purification by flash chromatography (CH₂Cl₂:CH₃OH, 49:1 to 47:3) afforded 11 as a white solid (286 mg, 87%, 2 steps): R_{f} 0.33 (CH₂Cl₂:CH₃OH 9:1); mp 212-214° C.; [α]²⁰_{D} +26.8° (c 1.25, CHCl₃). ¹H NMR (CDCl₃) δ: 0.77, 0.79, 0.84, 0.92, 1.00, 1.02, 1.68 (all s, each 3H, H-23, H-24, H-25, H-26, H-27, H-28, H-30), 2.38 (m, 1H, H-19), 2.64 (brs, 3H, 3×OH), 3.26 (dd, 1H, J=11.9 Hz, J=3.8 Hz, H-3), 3.54 (d, 1H, J=11.4 Hz, H′-5a), 3.65 (m, 1H, H′-3), 3.68 (m, 1H, H′-2), 3.93 (brs, 1H, H′-4), 3.94 (d, 1H, J=11.4 Hz, H′-5β), 4.34 (d, 1H, J=5.9 Hz, H′-1), 4.57 (brs, 1H, H-29α), 4.68 (brs, 1H, H-29β), 0.70-1.92 (all m, remaining protons). ¹³C NMR (CDCl₃) δ: 14.47, 15.98, 16.10, 16.39, 18.00, 18.30, 19.32, 20.96, 23.01, 25.13, 27.41, 28.20, 29.84, 34.26, 35.56, 37.03, 38.02, 38.22, 38.39, 40.00, 40.88, 42.82, 43.02, 47.98, 48.30, 50.39, 55.84, 64.83 (C′-5), 67.49 (C′-4), 71.62 (C′-3), 72.68 (C′-2), 84.59 (C-3), 99.53 (C′-1), 109.33 (C-29), 151.01 (C-20). HR-ESI-MS m/z 581.4163 [M+Na]⁺ (calcd for C₃₅H₅₈O₅Na, 581.4181).

3-O-β-D-Glucopyranoside of Betulin (Compound 12)

This compound was prepared from the acceptor 5 (500 mg, 1.03 mmol), and the donor 47 (1.15 g, 1.55 mmol) in the same manner as that described for compound 9. Purification by flash chromatography (CH₂Cl₂:CH₃OH, 49:1 to 47:3) afforded 12 as a white crystalline powder (406 mg, 65%, 2 steps): R_{f} 0.21 (CH₂Cl₂:CH₃OH 9:1); mp 192-194° C.; [α]_{D}+2.7° (c 0.58, CH₃OH). ¹H NMR (CD₃OD) δ: 0.84, 0.88, 1.02, 1.05, 1.08, 1.69 (all s, each 3H, H-23, H-24, H-25, H-26, H-27, H-30), 2.42 (m, 1H, H-19), 3.16 (dd, 1H, J=11.2 Hz, J=5.0 Hz, H-3), 3.18 (t, 1H, J=9.8 Hz, H′-2), 3.25 (m, 1H, H′-5), 3.28 (t, 1H, J=11.7 Hz, H′-4), 3.28 (d, 1H, J=11.7 Hz, H-28a), 3.28 (dd, 1H, J=11.9 Hz, J=5.1 Hz, H′-6a), 3.33 (t, 1H, J=9.8 Hz, H′-3), 3.74 (d, 1H, J=11.7 Hz, H-28β), 3.84 (dd, 1H, J=11.9 Hz, J=1.9 Hz, H′-6β), 4.31 (d, 1H, J=7.8 Hz, H′-1), 4.58 (brs, 1H, H-29a), 4.69 (brs, 1H, H-29β), 0.74-1.98 (all m, remaining protons). ¹³C NMR (CD₃OD) δ: 15.22, 16.54, 16.77, 16.82, 19.28, 19.38, 21.99, 26.62, 27.19, 28.17, 28.41, 30.37, 30.84, 35.10, 35.47, 38.02, 38.70, 40.00, 40.28, 42.16, 43.81, 48.53, 49.25, 50.03, 51.83, 57.10, 60.35 (C-28), 62.79 (C′-6), 71.64 (C′-4), 75.66 (C′-2), 77.68 (C′-5), 78.27 (C′-3), 90.79 (C-3), 106.74 (C′-1), 110.26 (C-29), 151.87 (C-20). HR-ESI-MS m/z 627.4218 [M+Na]⁺ (calcd for C₃₈H₈₀O₇Na, 627.4236).

3-O-α-L-Rhamnopyranoside of Betulin (Compound 13)

This compound was prepared from the acceptor 5 (252 mg, 0.52 mmol), and the donor 49 (484 mg, 0.78 mmol) in the same manner as that described for compound 9. Purification by flash chromatography (CH₂Cl₂:CH₃OH, 49:1 to 47:3) afforded 13 as a white crystalline powder (159 mg, 52%, 2 steps): R_{f} 0.29 (CH₂Cl₂:CH₃OH 9:1); mp>200° C.; [α]²⁰_{D}-20.3° (c 0.50, CH₃OH). ¹H NMR (CD₃OD) δ: 0.79, 0.88, 0.94, 1.02, 1.08 (all s, each 3H, H-23, H-24, H-25, H-26, H-27), 1.22 (d, 3H, J=6.3 Hz, H′-6), 1.69 (s, 3H, H-30), 2.42 (m, 1H, H-19), 3.07 (dd, 1H, J=11.3 Hz, J=4.6 Hz, H-3), 3.28 (d, 1H, J=10.9 Hz, H-28a), 3.36 (t, 1H, J=9.5 Hz, H′-4), 3.63 (dd, 1H, J=9.5 Hz, J=3.2 Hz, H′-3), 3.70 (m, 1H, H′-5), 3.74 (d, 1H, J=10.9 Hz, H-28β), 3.82 (brs, 1H, H′-2), 4.57 (brs, 1H, H-29a), 4.68 (brs, 1H, H-29β), 4.72 (brs, 1H, H′-1), 0.76-1.95 (all m, remaining protons). ¹³C NMR (CD₃OD) δ: 15.20, 16.51, 16.72, 16.77, 17.83 (C′-6), 19.34, 19.38, 21.98, 26.58, 26.76, 28.14, 28.61, 30.34, 30.82, 35.09, 35.40, 38.06, 38.68, 39.82, 40.15, 42.15, 43.82, 48.53, 49.24, 50.00, 51.77, 56.79, 60.33 (C-28), 69.88 (C′-5), 72.48 (C′-2), 72.50 (C′-3), 74.07 (C′-4), 90.36 (C-3), 104.43 (C′-1), 110.25 (C-29), 151.86 (C-20). HR-ESI-MS m/z 611.4266 [M+Na]⁺ (calculated for C₃₆H₆₀O₆Na, 611.4287).

3-O-α-D-Arabinopyranoside of Betulin (Compound 14)

This compound was prepared from the acceptor 5 (250 mg, 0.52 mmol), and the donor 51 (442 mg, 0.78 mmol) in the same manner as that described for compound 9. Purification by flash chromatography (CH₂Cl₂:CH₃OH, 49:1 to 47:3) afforded 14 as a white powder (196 mg, 66%, 2 steps): R_{f} 0.29 (CH₂Cl₂:CH₃OH 9:1); mp>200° C.; [α]²⁰_{D} +17.4 (c 0.25, CH₃OH). ¹H NMR(C₅D₅N) δ: 0.75, 0.84, 0.95, 1.05, 1.22, 1.75 (all s, each 3H, H-23, H-24, H-25, H-26, H-27, H-30), 2.61 (m, 1H, H-19), 3.42 (dd, 1H, J=11.4 Hz, J=4.2 Hz, H-3), 3.64 (d, 1H, J=10.1 Hz, H-28a), 3.80 (d, 1H, J=11.0 Hz, H′-5), 4.07 (d, 1H, J=10.1 Hz, H-28β), 4.18 (dd, 1H, J=8.7 Hz, J=2.8 Hz, H′-3), 4.32 (brs, 1H, H′-4), 4.34 (d, 1H, J=11.0 Hz, H′-5), 4.39 (t, 1H, J=7.9 Hz, H′-2), 4.70 (d, 1H, J=7.1 Hz, H′-1), 4.74 (brs, 1H, H-29a), 4.88 (brs, 1H, H-29β), 4.99 (brs, 3H, 3×OH), 0.72-2.42 (all m, remaining protons). ¹³C NMR (C₅D₅N) δ: 14.90, 16.12, 16.25, 16.91, 18.65, 19.26, 21.06, 23.86, 25.70, 27.54, 28.55, 29.98, 29.99, 30.02, 34.58, 34.87, 37.56, 38.80, 41.08, 41.21, 42.98, 48.35, 48.53, 49.13, 50.61, 56.20, 59.41 (C-28), 67.05 (C′-5), 69.61 (C′-4), 72.55 (C′-2), 74.79 (C′-3), 84.93 (C-3), 102.98 (C′-1), 109.93 (C-29), 151.25 (C-20). HR-ESI-MS m/z 587.4143 [M+Na]⁺ (calculated for C₃₅H₅₈O₆Na, 597.4131).

28-O-β-D-Glucopyranoside of Betulin (Compound 15)

This compound was prepared from the acceptor 6 (501 mg, 1.03 mmol), and the donor 47 (1.15 g, 1.55 mmol) in the same manner as that described for compound 9 except for the basic hydrolysis reaction time (overnight). Purification by flash chromatography (CH₂Cl₂:CH₃OH, 49:1 to 47:3) afforded 15 as a white powder (338 mg, 54%, 2 steps): R_{f} 0.21 (CH₂Cl₂:CH₃OH 9:1); mp>200° C.; [α]²⁰_{D}-12.8° (c 0.25, CH₃OH). ¹H NMR (CD₃OD) δ: 0.76, 0.87, 0.96, 1.01, 1.09, 1.69 (all s, each 3H, H-23, H-24, H-25, H-26, H-27, H-30), 2.46 (m, 1H, H-19), 3.13 (dd, 1H, J=11.1 Hz, J=4.9 Hz, H-3), 3.19 (t, 1H, J=8.4 Hz, H′-2), 3.28 (d, 1H, J=4.7 Hz, H′-5), 3.28 (d, 1H, J=6.0 Hz, H′-4), 3.36 (t, 1H, J=8.9 Hz, H′-3), 3.61 (d, 1H, J=9.5 Hz, H-28a), 3.68 (dd, 1H, J=11.8 Hz, J=5.0 Hz, H′-6a), 3.73 (d, 1H, J=9.5 Hz, H-28β), 3.89 (d, 1H, J=11.6 Hz, H′-6β), 4.22 (d, 1H, J=7.7 Hz, H′-1), 4.57 (brs, 1H, H-29a), 4.68 (brs, 1H, H-29β), 0.71-2.14 (all m, remaining protons). ¹³C NMR (CD₃OD) δ: 15.33, 16.18, 16.67, 16.75, 19.46, 19.50, 22.03, 26.66, 28.08, 28.40, 28.66, 30.69, 30.89, 35.51, 35.87, 38.32, 38.97, 40.00, 40.09, 42.18, 43.86, 46.96, 49.31, 50.17, 51.89, 56.85, 62.87 (C′-6), 68.91 (C-28), 71.77 (C′-4), 75.29 (C′-2), 77.96 (C′-5), 78.21 (C′-3), 79.70 (C-3), 105.35 (C′-1), 110.23 (C-29), 152.00 (C-20). HR-ESI-MS m/z 627.4229 [M+Na]⁺ (calculated for C₃₆H₆₀O₇Na, 627.4236).

28-O-α-L-Rhamnopyranoside of Betulin (Compound 16)

This compound was prepared from the acceptor 6 (250 mg, 0.52 mmol), and the donor 49 (480 mg, 0.77 mol) in the same manner as that described for compound 9 except for the basic hydrolysis reaction time (overnight). Purification by flash chromatography (CH₂Cl₂:CH₃OH, 49:1 to 47:3) afforded 16 as a white powder (203 mg, 67%, 2 steps): R_{f} 0.31 (CH₂Cl₂:CH₃OH 9:1); mp>200° C.; [α]²⁰_{D}-42.9° (c 0.83, CH₃OH). ¹H NMR (C₅D₅N) δ: 0.87, 0.95, 0.98, 1.03, 1.22, 1.73 (all s, each 3H, H-23, H-24, H-25, H-26, H-27, H-30), 1.73 (d, 3H, J=6.3 Hz, H′-6), 2.60 (m, 1H, H-19), 3.45 (m, 1H, H-3), 3.61 (d, 1H, J=9.4 Hz, H-28a), 3.83 (d, 1H, J=9.4 Hz, H-28β), 4.22 (c, 1H, H′-5), 4.33 (t, 1H, J=9.2 Hz, H′-4), 4.51 (dd, 1H, J=9.1 Hz, J=2.9 Hz, H′-3), 4.63 (brs, 1H, H′-2), 4.73 (brs, 1H, H-29a), 4.88 (brs, 1H, H-29β), 5.39 (brs, 1H, H′-1), 0.79-2.12 (all m, remaining protons). ¹³C NMR (C₅D₅N) δ: 14.89, 16.12, 16.37, 16.43, 18.74 (C′-6), 19.32, 21.00, 25.64, 27.55, 27.55, 28.31, 28.66, 30.33, 30.48, 34.59, 35.39, 37.46, 37.68, 39.27, 39.53, 41.15, 42.93, 47.31, 48.07, 49.07, 50.71, 55.83, 66.18 (C-28), 70.06 (C′-5), 72.45 (C′-2), 73.14 (C′-3), 73.94 (C′-4), 78.08 (C-3), 102.30 (C′-1), 110.11 (C-29), 150.89 (C-20). HR-ESI-MS m/z 611.4268 [M+Na]⁺ (calculated for C₃₆H₆₀O₆Na, 611.4287).

28-O-α-D-Arabinopyranoside of Betulin (Compound 17)

This compound was prepared from the acceptor 6 (250 mg, 0.52 mmol), and the donor 51 (469 mg, 0.77 mmol) in the same manner as that described for compound 9 except for the basic hydrolysis reaction time (overnight). Purification by flash chromatography (CH₂Cl₂:CH₃OH, 49:1 to 47:3) afforded 17 as a white crystalline powder (178 mg, 60%, 2 steps): R_{f} 0.43 (CH₂Cl₂:CH₃OH 9:1); mp 204-206° C.; [α]²⁰_{D}+4.6° (c 0.25, CH₃OH). ¹H NMR (DMSO-d₆) δ: 0.65, 0.76, 0.87, 0.93, 0.97, 1.63 (all s, each 3H, H-23, H-24, H-25, H-26, H-27, H-30), 2.40 (m, 1H, H-19), 2.96 (m, 1H, H-3), 2.99 (d, 1H, J=9.3 Hz, H-28a), 3.32 (m, 1H, H′-3), 3.33 (m, 1H, H′-2), 3.35 (d, 1H, J=11.8 Hz, H′-5a), 3.61 (m, 1H, H′-4), 3.66 (dd, 1H, J=11.8 Hz, J=3.4 Hz, H′-5b), 3.89 (d, 1H, J=9.3 Hz, H-28β), 4.06 (d, 1H, J=5.6 Hz, H′-1), 4.54 (brs, 1H, H-29a), 4.67 (brs, 1H, H-29β), 0.62-1.94 (all m, remaining protons). ¹³C NMR (DMSO-d₆) δ: 14.58, 15.67, 15.82, 15.90, 17.97, 18.76, 20.35, 24.74, 26.67, 27.18, 28.11, 29.29, 29.46, 33.76, 34.03, 36.68, 37.00, 38.25, 38.51, 40.45, 42.19, 46.60, 47.33, 48.33, 49.83, 54.86, 64.80 (C′-5), 66.33 (C-28), 67.40 (C′-4), 70.59 (C′-2), 72.60 (C′-3), 76.80 (C-3), 103.81 (C′-1), 109.77 (C-29), 150.17 (C-20). HR-ESI-MS m/z 597.4156 [M+Na] (calculated for C₃₅H₅₈O₆Na, 597.4131).

3-O-β-D-Glucopyranoside of Methyl Betulinate (Compound 18)

This compound was prepared from the acceptor 7 (251 mg, 0.53 mmol), and the donor 47 (593 mg, 0.80 mmol) in the same manner as that described for compound 9. Purification by flash chromatography (CH₂Cl₂:CH₃OH, 49:1 to 47:3) afforded 18 as a white crystalline powder (189 mg, 56%, 2 steps): R_{f} 0.24 (CH₂Cl₂:CH₃OH 9:1); mp 196-198° C., lit.²⁷ mp 197-200° C.; [α]²⁰_{D}-6.6° (c 0.50, CHCl₃), lit.²⁷ [α]ₚ-3° (c 0.38, CH₃OH). ¹H NMR (C₅D₅N) δ: 0.75, 0.94, 0.98, 1.02, 1.30, 1.72 (s, 3H, H-23, H-24, H-25, H-26, H-27, H-30), 3.30 (m, 1H, H-19), 3.40 (dd, 1H, J=11.7 Hz, J=4.3 Hz, H-3), 3.70 (s, 3H, COOCH₃), 4.01 (m, 1H, H′-5), 4.05 (t, 1H, J=8.3 Hz, H′-2), 4.23 (t, 1H, J=8.8 Hz, H′-4), 4.26 (t, 1H, J=8.5 Hz, H′-3), 4.41 (dd, 1H, J=11.6 Hz, J=5.4 Hz, H′-6a), 4.59 (dd, 1H, J=11.6 Hz, J=2.2 Hz, H′-6β), 4.72 (brs, 1H, H-29a), 4.88 (brs, 1H, H-29β), 4.95 (d, 1H, J=7.7 Hz, H′-1), 0.73-2.45 (all m, remaining protons). ¹³C NMR (C₅D₅N) δ: 14.80, 16.16, 16.32, 16.84, 18.42, 19.37, 21.05, 25.90, 26.76, 28.13, 30.04, 30.91, 32.31, 34.64, 37.08, 37.08, 38.49, 38.99, 39.63, 40.98, 42.67, 47.54, 49.75, 50.69, 51.33 (COOCH₃), 55.87, 56.77, 63.04 (C′-6), 71.84 (C′-4), 75.82 (C′-2), 78.35 (C′-5), 78.79 (C′-3), 88.81 (C-3), 106.92 (C′-1), 110.12 (C-29), 150.82 (C-20), 176.45 (C-28). HR-ESI-MS m/z 655.4164 [M+Na] (calculated for C₃₇H₆₀O₈Na, 655.4186).

3-O-α-L-Rhamnopyranoside of Methyl Betulinate (Compound 19)

This compound was prepared from the acceptor 7 (201 mg, 0.43 mmol), and the donor 49 (398 mg, 0.64 mmol) in the same manner as that described for compound 9. Purification by flash chromatography (CH₂Cl₂:CH₃OH, 49:1 to 47:3) afforded 19 as a white powder (176 mg, 67%, 2 steps): R_{f} 0.24 (CH₂Cl₂:CH₃OH 9:1); mp>200° C.; [α]²⁰_{D}-17.1° (c 0.42, CHCl₃). ¹H NMR (C₅D₅N) δ: 0.77 (s, 6H, H-25, H-26), 0.89, 0.96, 1.00 (all s, each 3H, H-23, H-24, H-27), 1.65 (d, 3H, J=5.4 Hz, H′-6), 1.72 (s, 3H, H-30), 3.14 (dd, 1H, J=11.7 Hz, J=4.3 Hz, H-3), 3.30 (m, 1H, H-19), 3.70 (s, 3H, COOCH₃), 4.29 (m, 1H, H′-4), 4.32 (m, 1H, H′-5), 4.49 (m, 1H, H′-3), 4.72 (brs, 1H, H′-2), 4.72 (brs, 1H, H-29a), 4.88 (brs, 1H, H-29β), 5.32 (brs, 1H, H′-1), 0.66-2.45 (all m, remaining protons). ¹³C NMR (C₅D₅N) δ: 14.77, 16.14, 16.27, 16.54, 18.52 (C′-6), 19.35, 21.05, 21.13, 25.88, 26.05, 28.13, 30.02, 30.90, 32.29, 33.71, 34.56, 37.07, 38.46, 38.80, 39.28, 40.96, 42.65, 47.53, 49.73, 50.66, 51.34 (COOCH₃), 55.61, 56.77, 69.87 (C′-5), 72.51 (C′-2), 72.91 (C′-3), 74.12 (C′-4), 88.51 (C-3), 104.42 (C′-1), 110.13 (C-29), 150.80 (C-20), 176.44 (C-28). HR-ESI-MS m/z 639.4223 [M+Na]⁺ (calculated for C₃₇H₆₀O₇Na, 639.4237).

3-O-α-D-Arabinopyranoside of Methyl Betulinate (Compound 20)

This compound was prepared from the acceptor 7 (200 mg, 0.42 mmol), and the donor 51 (387 mg, 0.64 mmol) in the same manner as that described for compound 9. Purification by flash chromatography (CH₂Cl₂:CH₃OH, 49:1 to 47:3) afforded 20 as a white powder (169 mg, 66%, 2 steps): R_{f} 0.24 (CH₂Cl₂:CH₃OH 9:1); mp>200° C.; [α]²⁰_{D} +22.7° (c 0.42, CHCl₃). ¹H NMR (CDCl₃) δ: 0.75, 0.81, 0.90, 0.93, 0.98, 1.68 (all s, each 3H, H-23, H-24, H-25, H-26, H-27, H-30), 3.00 (m, 1H, H-19), 3.02 (brs, 3H, 3×OH), 3.23 (dd, 1H, J=11.8 Hz, J=3.8 Hz, H-3), 3.52 (d, 1H, J=11.4 Hz, H′-5a), 3.66 (s, 3H, COOCH₃), 3.66 (m, 1H, H′-3), 3.70 (m, 1H, H′-2), 3.93 (m, 1H, H′-4), 3.95 (d, 1H, J=9.4 Hz, H′-5β), 4.31 (d, 1H, J=6.1 Hz, H′-1), 4.59 (brs, 1H, H-29a), 4.73 (brs, 1H, H-29β), 0.68-2.22 (all m, remaining protons). ¹³C NMR (CDCl₃) δ: 14.76, 16.09, 16.23, 16.54, 18.42, 19.51, 21.04, 23.15, 25.63, 28.32, 29.78, 30.73, 32.29, 34.44, 37.11, 37.18, 38.34, 38.37, 38.54, 40.85, 42.51, 47.10, 49.59, 50.63, 51.44 (COOCH₃), 56.02, 56.69, 65.10 (C′-5), 67.80 (C′-4), 71.69 (C′-3), 72.85 (C′-2), 84.81 (C-3), 99.79 (C′-1), 109.72 (C-29), 150.74 (C-20), 176.81 (C-28). HR-ESI-MS m/z 625.4073 [M+Na]⁺ (calculated for C₃₆H₅₈O₇Na, 625.4080).

3-O-β-D-Glucopyranoside of Betulinic Acid (Compound 21)

The acceptor 8 (107 mg, 0.22 mmol), and the donor 47 (239 mg, 0.32 mmol) were stirred in dry CH₂Cl₂ (10 mL) for 1 h with 4 Å MS. At this time, TMSOTf (3 μL, 0.01 mmol) was added under Ar while keeping rigorous anhydrous conditions. The reaction was usually performed in 30 min, then quenched by addition of Et₃N (50 μL). The solvents were evaporated under reduced pressure and the resulting residue was immediately dissolved in a NaOH 0.25 N solution of CH₃OH:THF:H₂O 1:2:1 (30 mL). The reaction mixture was stirred at room temperature for 2 h, dissolved in CH₂Cl₂ and washed with HCl 10% and brine. Once the solution was dried (MgSO₄), the solvents were evaporated under reduced pressure to give an oily residue. It was dissolved in a solution of PPh₃ (32 mg, 0.121 mmol) and pyrrolidine (34 μL, 0.403 mmol) in dry THF (1 mL), then Pd° (PPh₃)₄ (70 mg, 0.060 mmol), was added and the reaction was stirred overnight at room temperature. After evaporation of the solvent under reduced pressure, the residue was purified by flash chromatography (CH₂Cl₂:CH₃OH, 49:1 to 4:1) to give 21 as a white powder (63 mg, 47%, 3 steps): R_{f} 0.38 (CH₂Cl₂:CH₃OH 4:1); mp 234-236° C.; [α]²⁰_{D} +1.3° (c 0.33, CH₃OH). ¹H NMR (C₅D₅N) δ: 0.73, 0.97, 1.01, 1.09, 1.30, 1.77 (all s, each 3H, H-23, H-24, H-25, H-26, H-27, H-30), 3.41 (dd, 1H, J=11.6 Hz, J=4.0 Hz, H-3), 3.54 (m, 1H, H-19), 4.02 (m, 1H, H′-5), 4.05 (t, 1H, J=11.1 Hz, H′-2), 4.24 (m, 1H, H′-4), 4.26 (m, 1H, H′-3), 4.42 (dd, 1H, J=11.6 Hz, J=5.2 Hz, H′-6a), 4.60 (d, 1H, J=11.1 Hz, H′-6β), 4.75 (brs, 1H, H-29a), 4.93 (brs, 1H, H-29β), 4.95 (d, 1H, J=7.8 Hz, H′-1), 0.73-2.69 (all m, remaining protons). ¹³C NMR (C₅D₅N) δ: 14.84, 16.31, 16.35, 16.82, 18.44, 19.43, 21.15, 26.05, 26.76, 28.19, 30.25, 31.18, 32.85, 34.72, 37.11, 37.57, 38.56, 39.00, 39.63, 41.07, 42.83, 47.76, 49.71, 50.77, 55.88, 56.62, 63.03 (C′-6), 71.84 (C′-4), 75.82 (C′-2), 78.34 (C′-5), 78.78 (C′-3), 88.82 (C-3), 106.92 (C′-1), 109.95 (C-29), 151.29 (C-20), 178.87 (C-28). HR-ESI-MS m/z 641.4019 [M+Na]⁺ (calculated for C₃₆H₅₈O₈Na, 641.4029).

3-O-α-L-Rhamnopyranoside of betulinic acid (Compound 22)

This compound was prepared from the acceptor 8 (100 mg, 0.20 mmol), and the donor 49 (187 mg, 0.30 mmol) in the same manner as that described for compound 21. Purification by flash chromatography (CH₂Cl₂:CH₃OH, 49:1 to 4:1) afforded 22 as a white solid (50 mg, 41%, 3 steps): R_{f} 0.18 (CH₂Cl₂:CH₃OH 9:1); mp>200° C.; [α]²⁰_{D}-22.8° (c 0.42, CH₃OH). ¹H NMR (C₅D₅N) δ: 0.75, 0.76, 0.89, 1.02, 1.07 (all s, each 3H, H-23, H-24, H-25, H-26, H-27), 1.66 (d, 3H, J=5.0 Hz, H′-6), 1.77 (s, 3H, H-30), 3.16 (dd, 1H, J=11.5 Hz, J=4.0 Hz, H-3), 3.53 (m, 1H, H-19), 4.29 (m, 1H, H′-4), 4.31 (m, 1H, H′-5), 4.48 (m, 1H, H′-3), 4.58 (brs, 1H, H′-2), 4.75 (brs, 1H, H-29a), 4.93 (brs, 1H, H-29β), 5.33 (brs, 1H, H′-1), 0.67-2.71 (all m, remaining protons). ¹³C NMR (C₅D₅N) δ: 14.83, 16.28, 16.36, 16.54, 18.49, 18.53 (C′-6), 19.44, 21.18, 25.80, 26.06, 28.15, 30.26, 31.20, 32.86, 34.68, 37.13, 37.58, 38.56, 38.84, 39.30, 41.07, 42.84, 47.77, 49.73, 50.77, 55.65, 56.64, 69.88 (C′-5), 72.52 (C′-2), 72.93 (C′-3), 74.15 (C′-4), 88.53 (C-3), 104.42 (C′-1), 109.97 (C-29), 151.29 (C-20), 178.88 (C-28). HR-ESI-MS m/z 625.4057 [M+Na]⁺ (calculated for C₃₆H₅₈O₇Na, 625.4080).

3-O-α-D-Arabinopyranoside of Betulinic Acid (Compound 23)

This compound was prepared from the acceptor 8 (102 mg, 0.21 mmol), and the donor 51 (187 mg, 0.31 mmol) in the same manner as that described for compound 21. Purification by flash chromatography (CH₂Cl₂:CH₃OH, 49:1 to 4:1) afforded 23 as a white powder (60 mg, 50%, 3 steps): R_{f} 0.19 (CH₂Cl₂:CH₃OH 9:1); mp>200° C.; [α]²⁰_{D} +14.0° (c 1.00, CH₃OH). ¹H NMR (C₅D₅N) δ: 0.71, 0.81, 1.01, 1.07, 1.21, 1.78 (all s, each 3H, H-23, H-24, H-25, H-26, H-27, H-30), 3.42 (dd, 1H, J=11.6 Hz, J=4.0 Hz, H-3), 3.53 (m, 1H, H-19), 3.80 (d, 1H, J=11.0 Hz, H′-5a), 4.18 (dd, 1H, J=8.7 Hz, J=2.7 Hz, H′-3), 4.33 (brs, 1H, H′-4), 4.34 (d, 1H, J=11.0 Hz, H′-5β), 4.39 (t, 1H, J=7.9 Hz, H′-2), 4.67 (d, 1H, J=7.0 Hz, H′-1), 4.77 (brs, 1H, H-29a), 4.94 (brs, 1H, H-29β), 0.73-2.72 (all m, remaining protons). ¹³C NMR (C₅D₅N) δ: 14.80, 16.20, 16.33, 16.86, 18.62, 19.40, 21.16, 23.84, 26.04, 28.53, 30.22, 31.15, 32.83, 34.71, 37.29, 37.56, 38.53, 38.78, 38.81, 41.08, 42.81, 47.75, 49.72, 50.76, 56.25, 56.60, 67.02 (C′-5), 69.58 (C′-4), 72.51 (C′-2), 74.75 (C′-3), 84.93 (C-3), 102.97 (C′-1), 109.96 (C-29), 151.30 (C-20), 178.82 (C-28). HR-ESI-MS m/z 611.3908 [M+Na]⁺ (calculated for C₃₅H₅₆O₇Na, 611.3924).

3-Acetoxybetulinic Acid (Compound 24)

1.00 g of 3-acetoxybetulinal (2.27 mmol) was dissolved in 50 mL of t-BuOH, 10 mL of distilled THF and 15 mL of 2-methyl-2-butene. The solution was stirred and cooled with an iced-bath. Hence, 30 mL of freshly prepared solution of aqueous NaH₂PO₄/NaClO₂ (2.50 g/2.50 g in 30 mL of distilled water) was slowly added to the solution and the mixture was stirred 15 minutes at this temperature. After, the temperature of the mixture was raised to rt. and stirred for one hour. Finally, the mixture was poured into 50 mL of saturated NH₄Cl and extracted three times with CH₂Cl₂. The combined organic layers were dried over Na₂SO₄, filtered and evaporated under reduced pressure. Purification of the crude product by flash chromatography using isocratic 7% EtOAc in hexanes as eluent afforded 24 as a white solid (772 mg, 81%). I.R.: 2945, 1735 (C═O), 1696 (C═O), 1452, 1369, 1244 (C-0 ester), 1027, 979; ¹H NMR (CDCl₃): 4.74 (s br, 1H, H-29), 4.61 (s br, 1H, H-29), 4.47 (dd, 1H, J=10.40 Hz, J=5.60 Hz, H-3), 3.00 (m, 1H), 2.30-0.70 (25H), 2.04 (s, 3H), 1.69 (s, 3H), 0.97 (s, 3H), 0.93 (s, 3H), 0.85 (s, 3H), 0.84 (s, 3H), 0.83 (s, 3H); ¹³C NMR (CDCl₃): 182.19, 171.21, 150.51, 109.90, 81.09, 56.54, 55.55, 50.53, 49.40, 47.09, 42.56, 40.83, 38.56, 38.52, 37.95, 37.27, 37.19, 34.37, 32.30, 30.71, 29.84, 28.10, 25.58, 23.84, 21.48, 20.99, 19.50, 19.41, 18.31, 16.62, 16.33, 16.19, 14.81.

3-O-β-D-Galactopyranoside of Betulin (Compound 25)

This compound was prepared from the acceptor 2 (250 mg, 0.52 mmol), and the donor 52 (578 mg, 0.78 mmol) in the same manner as that described for compound 9. Purification by flash chromatography (CH₂Cl₂:CH₃OH, 49:1 to 47:3) afforded 25 as a white solid (60 mg, 19%, 2 steps). I.R.: 3373, 2920, 2853, 1457, 1353, 1246, 1145, 1029, 973, 876; ¹H NMR (Pyr-d5): 4.90 (m, 2H, H-1′, H-29), 4.75 (s, 1H, H-29), 4.62 (s, 1H, H-4′), 4.51 (m, 3H, H-6′ (2×), H-2′), 4.20 (m, 1H, H-3′), 4.16 (m, 1H, H-5′), 4.12 (m, 1H, H-28), 3.68 (m, 1H, H-28), 3.43 (m, 1H, H-3) 2.70-0.60 (25H), 1.78 (s, 3H), 1.33 (s, 3H), 1.10 (s, 3H), 0.99 (s, 3H), 0.98 (s, 3H), 0.80 (s, 3H); ¹³C NMR (Pyr-d5): 151.64, 110.33, 107.98, 89.14, 77.25, 75.91, 73.60, 70.72, 62.89, 59.82, 56.24, 51.02, 49.51, 48.94, 48.73, 43.37, 41.57, 40.05, 39.45, 37.95, 37.46, 35.26, 34.99, 30.78, 30.39, 28.52, 27.94, 27.27, 26.11, 21.45, 19.66, 18.87, 17.20, 16.75, 16.50, 15.33; HR-ESI-MS m/z 627.4214 [M+Na]⁺ (calculated for C₃₆H₆₀O₇Na, 627.4237).

3-O-β-D-Mannopyranoside of Betulin (Compound 26)

This compound was prepared from the acceptor 2 (261 mg, 0.54 mmol), and the donor 53 (600 mg, 0.81 mmol) in the same manner as that described for compound 9. Purification by flash chromatography (CH₂Cl₂:CH₃OH, 49:1 to 47:3) afforded 26 as a white powder (159 mg, 49%, 2 steps). I.R.: 3303, 2933, 2866, 1451, 1374, 1056, 1058, 978, 880, 679; ¹H NMR (Pyr-d5): 5.61 (br s, 1H, H-1′), 4.90 (d, 1H, J=2.20 Hz, H-29), 4.76 (s, 1H, H-29), 4.73 (m, 1H, H-4′), 4.64 (m, 1H, H-3′), 4.62 (m, 1H, H-6′), 4.57 (m, 1H, H-2′), 4.51 (m, 1H, H-5′), 4.45 (m, 1H, H-6′), 4.09 (d, 1H, J=11.16 Hz, H-28), 3.67 (d, 1H, J=10.72 Hz, H-28), 3.52 (dd, 1H, J=11.52 Hz, J=4.24 Hz, H-3), 2.70-0.60 (25H), 1.78 (s, 3H), 1.16 (s, 3H), 1.02 (s, 3H), 0.96 (s, 3H), 0.84 (s, 3H), 0.78 (s, 3H); ¹³C NMR (Pyr-d5): 151.65, 110.33, 98.12, 81.99, 76.39, 73.63, 73.40, 69.61, 63.80, 59.82, 56.17, 50.94, 49.49, 48.92, 48.72, 43.34, 41.54, 39.10, 38.81, 37.93, 37.62, 35.25, 34.90, 30.77, 30.40, 29.27, 27.92, 26.05, 22.60, 21.42, 19.66, 18.88, 17.15, 16.67, 16.50, 15.33; HR-ESI-MS m/z 627.4243 [M+Na]+ (calculated for C₃₆H₆₀O₇Na, 627.4237).

3-O-β-D-Xylopyranoside of Betulin (Compound 27)

This compound was prepared from the acceptor 2 (251 mg, 0.52 mmol), and the donor 54 (473 mg, 0.78 mmol) in the same manner as that described for compound 9. Purification by flash chromatography (CH₂Cl₂:CH₃OH, 49:1 to 47:3) afforded 27 as a white solid (81 mg, 27%, 2 steps). I.R.: 3343, 2937, 2866, 1450, 1374, 1242, 1161, 1039, 974, 880, 635; ¹H NMR (Pyr-d5): 4.90 (d, 1H, J=2.08 Hz, H-29), 4.88 (d, 1H, J=7.60 Hz, H-1′), 4.75 (s, 1H, H-29), 4.40 (m, 1H, H-5′), 4.26 (m, 1H, H-4′), 4.19 (m, 1H, H-3′), 4.11 (d, 1H, J=10.56 Hz, H-28), 4.06 (m, 1H, H-2′), 3.80 (m, 1H, H-5′), 3.68 (d, 1H, J=10.44 Hz, H-28), 3.41 (dd, 1H, J=11.68 Hz, J=4.36 Hz, H-3), 2.70-0.70 (25H), 1.77 (s, 3H), 1.33 (s, 3H), 1.09 (s, 3H), 1.02 (s, 3H), 0.99 (s, 3H), 0.83 (s, 3H); ¹³C NMR (Pyr-d5): 151.06, 110.35, 108.08, 89.07, 79.04, 75.97, 71.64, 67.54, 59.76, 56.24, 51.03, 49.50, 48.93, 48.72, 43.35, 41.58, 40.10, 39.41, 37.94, 37.51, 35.26, 34.96, 30.76, 30.41, 28.49, 27.94, 27.35, 26.06, 21.43, 19.64, 18.86, 17.20, 16.76 16.51, 15.29; HR-ESI-MS m/z 597.4146 [M+Na] (calculated for C₃₅H₅₈O₆Na, 597.4131).

3-O-β-D-Galactopyranoside of Betulinic Acid (Compound 28)

This compound was prepared from the acceptor 8 (207 mg, 0.42 mmol), and the donor 52 (467 mg, 0.63 mmol) in the same manner as that described for compound 21. Purification by flash chromatography (CH₂Cl₂:CH₃OH, 49:1 to 4:1) afforded 28 as a white solid (111 mg, 43%; 3 steps). I.R.: 3325, 2936, 2864, 1687, 1449, 1375, 1214, 1152, 1056, 976, 879; ¹H NMR (Pyr-d5): 4.96 (s, 1H, H-29), 4.90 (d, 1H, J=7.56 Hz, H-1′), 4.77 (s, 1H, H-29), 4.63 (m, 1H, H-4′), 4.50 (m, 3H, H-6′ (2×), H-2′), 4.21 (m, 1H, H-3′), 4.15 (m, 1H, H-5′), 3.56 (m, 1H, H-19), 3.42 (m, 1H, H-3) 2.80-0.60 (24H), 1.80 (s, 3H), 1.32 (s, 3H), 1.12 (s, 3H), 1.03 (s, 3H), 0.96 (s, 3H), 0.76 (s, 3H); ¹³C NMR (Pyr-d5): 179.32, 151.69, 110.35, 107.95, 89.11, 77.25, 75.91, 73.62, 70.66, 62.84, 57.02, 56.31, 51.20, 50.13, 48.16, 43.22, 41.45, 40.04, 39.44, 38.96, 37.98, 37.50, 35.13, 33.27, 31.59, 30.65, 28.51, 27.26, 26.46, 21.56, 19.84, 18.83, 17.18, 16.76, 16.74, 15.25; HR-ESI-MS m/z 641.4005 [M+Na]⁺ (calculated for C₃₆H₅₈O₈Na, 641.4029).

3-O-β-D-Mannopyranoside of Betulinic Acid (Compound 29)

This compound was prepared from the acceptor 8 (201 mg, 0.40 mmol), and the donor 53 (445 mg, 0.60 mmol) in the same manner as that described for compound 21. Purification by flash chromatography (CH₂Cl₂:CH₃OH, 49:1 to 4:1) afforded 29 as a white solid (58 mg, 23%, 3 steps). I.R.: 3382, 2944, 1686, 1440, 1376, 1241, 1106, 1058, 1028, 975, 881, 814; ¹H NMR (Pyr-d5): 5.60, s br, 1H, H-1′), 4.96 (s br, 1H, H-29), 4.78 (s br, 1H, H-29), 4.75 (m, 2H, H-4′), 4.63 (m, 2H, H-3′, H-6′),), 4.57 (s br, 1H, H-2′), 4.49 (m, 2H, H-5′, H-6′), 3.55 (m, 1H, H-19), 3.53 (m, 1H, H-3), 3.00-0.50 (24H), 1.80 (s, 3H), 1.16 (s, 3H), 1.04 (s, 3H), 1.02 (s, 3H), 0.81 (s, 3H), 0.74 (s, 3H); ¹³C NMR (Pyr-d5): 179.29, 151.73, 110.35, 98.10, 81.95, 76.41, 73.67, 73.41, 69.63, 63.82, 57.01, 56.24, 51.12, 50.11, 50.06, 43.19, 41.43, 39.09, 38.92, 38.81, 37.98, 37.66, 35.04, 33.24, 31.57, 30.62, 29.26, 26.42, 22.56, 21.52, 19.82, 18.87, 17.13, 16.74, 16.65, 15.25; HR-ESI-MS m/z 641.4017 [M+Na] (calculated for C₃₆H₅₈O₈Na, 641.4029).

3-O-β-D-Xylopyranoside of betulinic acid (Compound 30)

This compound was prepared from the acceptor 8 (200 mg, 0.40 mmol), and the donor 54 (364 mg, 0.60 mmol) in the same manner as that described for compound 21. Purification by flash chromatography (CH₂Cl₂:CH₃OH 49:1 to 4:1) afforded 30 as a white solid (138 mg, 58%, 3 steps). I.R.: 3376, 2931, 2865, 1687, 1638, 1453, 1375, 1161, 1046, 974, 882; ¹H NMR (Pyr-d5): 4.96 (s, 1H, H-29), 4.87 (d, 1H, J=7.04 Hz, H-1′), 4.78 (s, 1H, H-29), 4.39 (m, 1H, H-5′), 4.26 (m, 1H, H-4′), 4.20 (m, 1H, H-3′), 4.05 (m, 1H, H-2′), 3.80 (m, 1H, H-5′), 3.56 (m, 1H, H-19), 3.40 (m, 1H, H-3), 2.80-0.70 (24H), 1.79 (s, 3H), 1.32 (s, 3H), 1.11 (s, 3H), 1.04 (s, 3H), 0.99 (s, 3H), 0.78 (s, 3H); ¹³C NMR (Pyr-d5): 179.27, 151.67, 110.39, 108.09, 89.06, 79.05, 75.98, 71.64, 67.54, 57.01, 56.31, 51.20, 50.12, 48.16, 43.20, 41.46, 40.09, 39.45, 38.94, 37.97, 37.56, 35.11, 33.24, 31.57, 30.64, 28.48, 27.35, 26.44, 21.56, 19.81, 18.85, 17.18, 16.75 (2×), 15.22; HR-ESI-MS m/z 587.3961 [M—H]⁺ (calculated for C₃₅H₅₅O₇, 587.3953).

Allobetulin (Compound 31)

This compound was prepared as previously reported (Lavoie, S.; Pichette, A.; Garneau, F.-X.; Girard, M.; Gaudet, D. Synthetic Communication, 2001, 31(10), 1565-1571) following this procedure: 5.00 g of betulin (2) (11.29 mmol) dissolved in 500 mL of CH₂Cl₂ with a mixture of Fe(NO₃)₃:SiO₂ (1:4) grinded on a mortar (9.13 g:36.50 g, 22.58 mmol of Fe(NO₃)₃) were refluxed for 45 minutes. The solution was then filtered and washed with CH₂Cl₂ and evaporated under reduced pressure. The crude product was purified by flash chromatography on silica gel using Hexanes:EtOAc (9:1 to 4:1) as eluent to afford 31 as a white solid (3.60 g, 72%). I.R.: 3452, 2926, 2863, 1450, 1386, 1264, 1180, 1138, 1088, 1042, 1005, 987, 971, 887, 810, 768, 737; ¹H NMR (CDCl₃): 3.76 (d, 1H, J=7.56 Hz, H-28), 3.52 (s, 1H, H-19), 3.43 (d, 1H, J=7.80 Hz, H-28), 3.19 (m, 1H, H-3), 2.00-1.00 (24H), 0.96 (s, 6H), 0.92 (s, 3H), 0.90 (s, 3H), 0.83 (s, 3H), 0.79 (s, 3H), 0.76 (s, 3H); ¹³C NMR (CDCl₃): δ8.06, 79.08, 71.39, 55.60, 51.20, 46.95, 41.60, 40.83, 40.73, 39.04, 39.01, 37.38, 36.87, 36.39, 34.26, 34.03, 32.83, 28.94, 28.11, 27.54, 26.58, 26.57, 26.39, 24.68, 21.11, 18.38, 16.62, 15.84, 15.52, 13.64.

28-Oxyallobetulin (Compound 32)

500 mg of betulinic acid (3) (1.00 mmol) was stirred under refluxed in 25 mL of CH₂Cl₂ with a mixture of FeCl₃:SiO₂ (1:4) grinded on a mortar (0.50 g:1.95 g, 3.00 mmol of FeCl₃) for 3 h. The mixture was then filtered on celite and washed with CH₂Cl₂, evaporated and dissolved in a 1:2:1 MeOH:THF:H₂O (50 mL) who was refluxed with 1.00 g of NaOH (25 mmol) overnight. Then, 25 mL of CH₂Cl₂ was added and the solution was neutralised with HCl 10% until pH 4˜5 and extracted with CH₂Cl₂ three times with portions of 50 mL. Combined organic layers dried over Na₂S₂O₄, filtered and evaporated, afforded crude product who was purified by flash chromatography on silica gel with CH₂Cl₂:CH₃OH (99:1 to 97:3) as eluent to afford 32 as a white solid (417 mg, 91%, 2 steps). I.R.: 3377, 2941, 1760, 1446, 1388, 1153, 1119, 1045, 966, 922, 733; ¹H NMR (CDCl₃): 3.93 (s, 1H, H-19), 3.20 (dd, 1H, J=11.24 Hz, J=4.88 Hz, H-3), 2.00-0.50 (24H), 1.02 (s, 3H), 0.96 (s, 3H), 0.95 (s, 3H), 0.90 (s, 3H), 0.86 (s, 3H), 0.83 (s, 3H), 0.75 (s, 3H); ¹³C NMR (CDCl₃): 179.86, 85.99, 78.89, 55.49, 51.23, 46.70, 46.09, 40.55, 39.91, 38.93, 38.87, 37.25, 36.00, 33.71, 33.54, 32.31, 31.93, 28.74, 27.94, 27.88, 27.35, 26.51, 25.54, 23.95, 20.87, 18.14, 16.53, 15.51, 15.34, 13.65.

3-O-β-D-Glucopyranoside of Allobetulin (Compound 33)

This compound was prepared from the acceptor 31 (80 mg, 0.18 mmol), and the donor 47 (200 mg, 0.27 mmol) in the same manner as that described for compound 9. Purification by flash chromatography (CH₂Cl₂:CH₃OH, 49:1 to 47:3) afforded 33 as a white solid (82 mg, 75%, 2 steps). I.R.: 3350, 2923, 2865, 1448, 1387, 1374, 1358, 1304, 1162, 1072, 1035, 1022, 893, 766; ¹H NMR (Pyr-d5): 4.98 (d, 1H, J=7.75 Hz, H-1′), 4.64 (m, 1H, H-6′), 4.45 (m, 1H, H-6′), 4.26 (m, 2H, H-3′ and H-4′), 4.07 (m, 1H, H-5′), 4.04 (m, 1H, H-2′), 3.87 (d, 1H, J=7.75 Hz, H-28), 3.68 (s, 1H, H-19), 3.51 (d, 1H, J=7.60 Hz, H-28), 3.41 (m, 1H, H-3), 2.28 (m, 1H, H-2), 1.87 (m, 1H, H-2), 1.70-0.70 (22H), 1.34 (s, 3H), 1.07 (s, 3H), 1.03 (s, 3H), 0.97 (s, 3H), 0.88 (s, 3H), 0.84 (s, 3H), 0.79 (s, 3H); ¹³C NMR (Pyr-d5): 107.36, 89.21, 88.23, 79.18, 78.77, 76.21, 72.27, 71.63, 63.48, 56.37, 51.63, 47.52, 42.02, 41.31, 41.18, 40.04, 39.49, 37.51, 37.32, 36.92, 34.89, 34.61, 33.55, 29.60, 28.51, 27.18, 27.15, 27.11, 26.93, 24.97, 21.67, 18.79, 17.25, 17.07, 16.18, 14.05; HR-ESI-MS m/z 627.4220 [M+Na]⁺ (calculated for C₃₆H₆₀O₇Na, 627.4237).

3-O-α-L-Rhamnopyranoside of Allobetulin (Compound 34)

This compound was prepared from the acceptor 31 (100 mg, 0.23 mmol), and the donor 49 (214 mg, 0.35 mmol) in the same manner as that described for compound 9. Purification by flash chromatography (CH₂Cl₂:CH₃OH, 49:1 to 47:3) afforded 34 as a white solid (110 mg, 83%, 2 steps). I.R.: 3408, 2926, 1448, 1386, 1130, 1106, 1051, 974, 811; ¹H NMR (Pyr-d5): 5.36 (d, 1H, J=1.16 Hz, H-1′), 4.61 (m, 1H, H-2′), 4.50 (m, 1H, H-3′), 4.36 (m, 1H, H-5′), 4.34 (m, 1H, H-4′), 3.87 (d, 1H, J=7.40 Hz, H-28), 3.68 (s, 1H, H-19), 3.51 (d, 1H, J=7.80 Hz, H-28), 2.00 (m, 1H, H-2), 1.90-0.60 (23H), 1.71 (d, 3H, J=5.72 Hz, H-6′), 1.09 (s, 3H), 0.94 (s, 3H), 0.94 (s, 3H), 0.89 (s, 3H), 0.85 (s, 3H), 0.83 (s, 3H), 0.81 (s, 3H); ¹³C NMR (Pyr-d5): 104.89, 88.87, 88.22, 74.52, 73.33, 72.90, 71.62, 70.25, 56.11, 51.60, 47.50, 42.00, 41.29, 41.15, 39.70, 39.32, 37.49, 37.30, 36.91, 34.88, 34.53, 33.54, 29.58, 28.50, 27.13, 27.09, 26.90, 26.44, 24.96, 21.67, 18.92, 18.87, 17.01, 16.94, 16.16, 14.01; HR-ESI-MS m/z 611.4267 [M+Na]⁺ (calculated for C₃₆H₆₀O₆Na, 611.4288).

3-O-α-D-Arabinopyranoside of Allobetulin (Compound 35)

This compound was prepared from the acceptor 31 (100 mg, 0.23 mmol), and the donor 51 (209 mg, 0.35 mmol) in the same manner as that described for compound 9. Purification by flash chromatography (CH₂Cl₂:CH₃OH, 49:1 to 47:3) afforded 35 as a white solid (103 mg, 79%, 2 steps). I.R.: 3343, 2939, 2926, 2871, 2855, 1450, 1386, 1137, 1290, 1252, 1069, 1033, 1001, 939, 767, 714; ¹H NMR (Pyr-d5): 4.76 (d, 1H, J=7.12 Hz, H-1′), 4.46 (m, 1H, H-2′), 4.41 (m, 1H, H-5′), 4.38 (m, 1H, H-4′), 4.23 (m, 1H, H-3′), 3.88 (m, 1H, H-5′), 3.85 (d, 1H, J=6.76 Hz, H-28), 3.69 (s, 1H, H-19), 3.51 (d, 1H, J=7.68 Hz, H-28), 3.46 (dd, 1H, J=12.40 Hz, J=4.56 Hz, H-3), 2.03 (m, 1H, H-2), 1.80-0.60 (24H), 1.22 (s, 3H), 1.08 (s, 3H), 0.96 (s, 3H), 0.88 (s, 6H), 0.85 (s, 3H), 0.77 (s, 3H); ¹³C NMR (Pyr-d5): 103.39, 88.21, 85.19, 75.21, 72.96, 71.63, 70.03, 67.49, 56.74, 51.61, 47.51, 42.00, 41.30, 41.19, 39.26, 39.01, 37.69, 37.30, 36.90, 34.85, 34.58, 33.54, 29.57, 28.90, 27.12, 27.09, 26.90, 24.95, 24.16, 21.68, 18.96, 17.29, 16.95, 16.16, 14.00; HR-ESI-MS m/z 597.4130 [M+Na] (calculated for C₃₅H₅₈O₆Na, 597.4131).

3-O-β-D-Galactopyranoside of Allobetulin (Compound 36)

This compound was prepared from the acceptor 31 (100 mg, 0.23 mmol), and the donor 52 (214 mg, 0.35 mmol) in the same manner as that described for compound 9. Purification by flash chromatography (CH₂Cl₂:CH₃OH, 49:1 to 47:3) afforded 36 as a white solid (91 mg, 67%, 2 steps). I.R.: 3407, 2941, 2868, 1641, 1449, 1386, 1140, 1056, 978, 667; ¹H NMR (Pyr-d5): 4.92, (d, 1H, J=7.75 Hz, H-1′), 4.63 (d, 1H, J=3.04 Hz, H-4′), 4.53 (m, 2H, H-6′), 4.50 (m, 1H, H-2′), 4.22 (m, 1H, H-3′), 4.17 (m, 1H, H-5′), 3.87 (d, 1H, J=7.98 Hz, H-28), 3.68 (s, 1H, H-19), 3.51 (d, 1H, J=7.75 Hz, H-28), 3.41 (m, 1H, H-3), 2.32 (m, 1H, H-2), 1.92 (m, 1H, H-2), 1.70-0.70 (22H), 1.33 (s, 3H), 1.09 (s, 3H), 1.00 (s, 3H), 0.97 (s, 3H), 0.88 (s, 3H), 0.85 (s, 3H), 0.80 (s, 3H); ¹³C NMR (Pyr-d5): 107.57, 88.67, 87.82, 76.87, 75.48, 73.18, 71.22, 70.30, 62.49, 55.97, 51.23, 47.11, 41.60, 40.89, 40.77, 39.65, 39.12, 37.10, 36.90, 36.50, 34.47, 34.19, 33.14, 29.17, 28.09, 26.86, 26.72, 26.70, 26.50, 24.54, 21.25, 18.36, 16.79, 16.66, 15.76, 13.63; HR-ESI-MS m/z 627.4215 [M+Na]⁺ (calculated for C₃₆H₆₀O₇Na, 627.4237).

3-O-α-D-Mannopyranoside of Allobetulin (Compound 37)

This compound was prepared from the acceptor 31 (100 mg, 0.23 mmol), and the donor 53 (214 mg, 0.35 mmol) in the same manner as that described for compound 9. Purification by flash chromatography (CH₂Cl₂:CH₃OH, 49:1 to 47:3) afforded 37 as a white solid (121 mg, 89%, 2 steps). I.R.: 3364, 2924, 2868, 1443, 1386, 1123, 1069, 1033, 811, 713; ¹H NMR (Pyr-d5): 5.62 (d, 1H, J=1.17 Hz, H-1′), 4.76 (m, 1H, H-4′), 4.65 (m, 1H, H-3′), 4.63 (m, 1H, H-6′), 4.59 (m, 1H, H-2′), 4.50 (m, 1H, H-5′), 4.48 (m, 1H, H-6′), 3.87 (d, 1H, J=7.75 Hz, H-28), 3.68 (s, 1H, H-19), 3.51 (d, 1H, J=7.60 Hz, H-28), 3.51 (m, 1H, H-3), 1.84 (m, 1H, H-2), 1.70-0.70 (23H), 1.18, (s, 3H), 1.08 (s, 3H), 0.91 (s, 3H), 0.86 (s, 3H), 0.85 (s, 3H), 0.84 (s, 3H), 0.77 (s, 3H); ¹³C NMR (Pyr-d5): 98.09, 88.21, 81.85, 76.43, 73.67, 73.42, 71.62, 69.60, 63.81, 56.33, 51.55, 47.51, 42.02, 41.29, 41.16, 39.13, 38.88, 37.68, 37.30, 36.91, 34.85, 34.51, 33.54, 29.58, 29.26, 27.13, 27.08, 26.89, 24.95, 22.55, 21.65, 18.82, 17.17, 17.00, 16.16, 14.05; HR-ESI-MS m/z 627.4221 [M+Na]⁺ (calculated for C₃₆H₆₀O₇Na, 627.4237).

3-O-β-D-Xylopyranoside of Allobetulin (Compound 38)

This compound was prepared from the acceptor 31 (100 mg, 0.23 mmol), and the donor 54 (209 mg, 0.35 mmol) in the same manner as that described for compound 9. Purification by flash chromatography (CH₂Cl₂:CH₃OH, 49:1 to 47:3) afforded 38 as a white solid (110 mg, 85%, 2 steps). I.R.: 3250, 2923, 1441, 1385, 1165, 1086, 1032, 969, 892, 767; ¹H NMR (Pyr-d5): 4.88 (d, 1H, J=7.60 Hz, H-1′), 4.43, (m, 1H, H-5′), 4.29 (m, 1H, H-4′), 4.22 (m, 1H, H-3′), 4.07 (m, 1H, H-2′), 3.87 (d, 1H, J=8.18 Hz, H-28), 3.82 (m, 1H, H-5′), 3.68 (s, 1H, H-19), 3.52 (d, 1H, J=8.04 Hz, H-28), 3.38 (m, 1H, H-3), 2.24 (m, 1H, H-2), 1.95 (m, 1H, H-2), 1.70-0.70 (22H), 1.33 (s, 3H), 1.09 (s, 3H), 1.02 (s, 3H), 0.96 (s, 3H), 0.88 (s, 3H), 0.85 (s, 3H), 0.79 (s, 3H); ¹³C NMR (Pyr-d5): 108.13, 89.01, 88.21, 79.06, 75.98, 71.65, 71.63, 67.56, 56.40, 51.66, 47.51, 42.00, 41.30, 41.18, 40.12, 39.54, 37.56, 37.30, 36.91, 34.86, 34.60, 33.54, 29.58, 28.45, 27.35, 27.13, 27.09, 26.91, 24.96, 21.67, 18.78, 17.19, 17.09, 16.18, 14.01; HR-ESI-MS m/z 597.4144 [M+Na]⁺ (calcd for C₃₆H₅₈O₆, 597.4131).

3-O-β-D-Glucopyranoside of 28-Oxyallobetulin (Compound 39)

This compound was prepared from the acceptor 32 (80 mg, 0.18 mmol), and the donor 47 (200 mg, 0.27 mmol) in the same manner as that described for compound 9. Purification by flash chromatography (CH₂Cl₂:CH₃OH, 49:1 to 47:3) afforded 39 as a white solid (56 mg, 50%, 2 steps). I.R.: 3388, 2943, 2869, 1766, 1447, 1388, 1375, 1304, 1154, 1072, 1016, 969, 923, 532; ¹H NMR (Pyr-d5): 4.98 (d, 1H, J=7.75 Hz, H-1′), 4.64 (m, 1H, H-6′), 4.45 (m, 1H, H-6′), 4.26 (m, 2H, H-3′ and H-4′), 4.08 (m, 1H, H-2′), 4.06 (s, 1H, H-19), 4.04 (m, 1H, H-5′), 3.40 (m, 1H, H-3), 2.28 (m, 1H, H-2), 2.00 (m, 1H, H-16), 1.86 (m, 2H, H-2 and H-18), 1.70-0.70 (20H), 1.32 (s, 3H), 1.04 (s, 3H), 1.00 (s, 3H), 0.93 (s, 3H), 0.90 (s, 3H), 0.78 (s, 3H), 0.75 (s, 3H); ¹³C NMR (Pyr-d5): 179.92, 107.36, 89.16, 86.25, 79.16, 78.77, 76.19, 72.25, 63.48, 56.33, 51.72, 47.18, 46.60, 41.10, 40.55, 40.00, 39.47, 37.46, 36.88, 34.40, 34.11, 33.12, 32.44, 29.19, 28.65, 28.47, 27.14, 26.97, 26.38, 24.05, 21.51, 18.66, 17.19, 17.07, 15.86, 14.12; HR-ESI-MS m/z 641.4038 [M+Na]⁺ (calculated for C₃₆H₅₈O₈Na, 641.4029).

3-O-α-L-Rhamnopyranoside of 28-Oxyallobetulin (Compound 40)

This compound was prepared from the acceptor 32 (100 mg, 0.22 mmol), and the donor 49 (205 mg, 0.33 mmol) in the same manner as that described for compound 9. Purification by flash chromatography (CH₂Cl₂:CH₃OH, 49:1 to 47:3) afforded 40 as a white solid (92 mg, 70%, 2 steps). I.R.: 3310, 2935, 1757, 1443, 1387, 1146, 1117, 1053, 965, 921, 810; ¹H NMR (Pyr-d5): 5.36 (d, 1H, J=1.16 Hz, H-1′), 4.62 (m, 1H, H-2′), 4.53 (m, 1H, H-3′), 4.37 (m, 1H, H-5′), 4.35 (m, 1H, H-4′), 4.07 (s, 1H, H-19), 3.17 (m, 1H, H-3), 2.00 (m, 1H, H-2), 2.00 (m, 1H, H-16), 1.87 (m, 1H, H-18), 1.80-0.60 (21H), 1.72 (d, 3H, J=5.72 Hz, H-6′), 1.04 (s, 3H), 0.93 (s, 3H), 0.92 (s, 3H), 0.87 (s, 3H), 0.80 (s, 3H), 0.79 (s, 3H), 0.76 (s, 3H); ¹³C NMR (Pyr-d5): 179.94, 104.94, 88.84, 86.26, 74.53, 73.35, 72.92, 70.28, 56.10, 51.72, 47.19, 46.59, 41.08, 40.56, 39.69, 39.31, 37.47, 36.88, 34.34, 34.12, 33.13, 32.45, 29.20, 28.66, 28.48, 26.97, 26.43, 26.39, 24.06, 21.55, 18.95, 18.77, 17.05, 16.91, 15.87, 14.10; HR-ESI-MS m/z 625.4055 [M+Na]⁺ (calculated for C₃₆H₅₈O₇Na, 625.4080).

3-O-α-D-Arabinopyranoside of 28-oxyallobetulin (Compound 41)

This compound was prepared from the acceptor 32 (250 mg, 0.55 mmol), and the donor 51 (500 mg, 0.82 mmol) in the same manner as that described for compound 9. Purification by flash chromatography (CH₂Cl₂:CH₃OH, 49:1 to 47:3) afforded 41 as a white solid (26 mg, 20%, 2 steps). I.R.: 3280, 2941, 2921, 1757, 1442, 1386, 1360, 1137, 1068, 1002, 965, 945, 921; ¹H NMR (Pyr-d5): 4.75 (d, 1H, J=7.12 Hz, H-1′), 4.45 (m, 1H, H-2′), 4.41 (m, 1H, H-5′), 4.38 (m, 1H, H-4′), 4.23 (m, 1H, H-3′), 4.07 (s, 1H, H-19), 3.85 (d, 1H, J=12.64 Hz, H-5′), 3.43 (m, 1H, H-3), 2.20-0.70 (24H), 1.24 (s, 3H), 1.03 (s, 3H), 0.93 (s, 3H), 0.88 (s, 3H), 0.86 (s, 3H), 0.78 (s, 3H), 0.73 (s, 3H); ¹³C NMR (Pyr-d5): 179.95, 103.33, 86.23, 85.07, 75.20, 72.94, 70.01, 67.47, 56.71, 51.71, 47.18, 46.57, 41.10, 40.55, 39.24, 38.98, 37.65, 36.84, 34.38, 34.09, 33.10, 32.42, 29.16, 28.86, 28.63, 26.96, 26.37, 24.10, 24.03, 21.54, 18.84, 17.23, 16.96, 15.85, 14.06; HR-ESI-MS m/z 611.3935 [M+Na]⁺ (calculated for C₃₅H₅₆O₇Na, 611.3924).

3-O-β-D-Galactopyranoside of 28-Oxyallobetulin (Compound 42)

This compound was prepared from the acceptor 32 (100 mg, 0.22 mmol), and the donor 52 (245 mg, 0.33 mmol) in the same manner as that described for compound 9. Purification by flash chromatography (CH₂Cl₂:CH₃OH, 49:1 to 47:3) afforded 42 as a white solid (83 mg, 61%, 2 steps). I.R.: 3378, 2935, 1758, 1446, 1389, 1153, 1055, 966, 922, 756; ¹H NMR (Pyr-d5): 4.91, (d, 1H, J=7.68 Hz, H-1′), 4.63 (d, 1H, J=3.04 Hz, H-4′), 4.53 (m, 2H, H-6′), 4.51 (m, 1H, H-2′), 4.22 (m, 1H, H-3′), 4.17 (m, 1H, H-5′), 4.07 (s, 1H, H-19), 3.40 (m, 1H, H-3), 2.32 (m, 1H, H-2), 2.01 (m, 1H, H-16), 1.90 (m, 1H, H-2), 1.88 (m, 1H, H-18), 1.70-0.70 (20H), 1.32 (s, 3H), 1.04 (s, 3H), 0.97 (s, 3H), 0.93 (s, 3H), 0.90 (s, 3H), 0.78 (s, 3H), 0.75 (s, 3H); ¹³C NMR (Pyr-d5): 179.94, 107.99, 89.02, 86.25, 77.29, 75.88, 73.58, 70.72, 62.91, 56.36, 51.75, 47.18, 46.59, 41.10, 40.56, 40.04, 39.52, 37.49, 36.88, 34.41, 34.12, 33.13, 32.44, 29.19, 28.66, 28.47, 27.24, 26.99, 26.39, 24.05, 21.53, 18.67, 17.16, 17.10, 15.87, 14.12; HR-ESI-MS m/z 641.4037 [M+Na]⁺ (calculated for C₃₆H₅₈O₈Na, 641.4029).

3-O-α-D-Mannopyranoside of 28-oxyallobetulin (Compound 43)

This compound was prepared from the acceptor 32 (100 mg, 0.22 mmol), and the donor 53 (245 mg, 0.33 mmol) in the same manner as that described for compound 9. Purification by flash chromatography (CH₂Cl₂:CH₃OH, 49:1 to 47:3) afforded 43 as a white solid (62 mg, 46%, 2 steps). I.R.: 3330, 2940, 1757, 1443, 1388, 1119, 1067, 965, 921; ¹H NMR (Pyr-d5): 5.62 (d, 1H, J=1.08 Hz, H-1′), 4.76 (m, 1H, H-4′), 4.65 (m, 1H, H-3′), 4.63 (m, 1H, H-6′), 4.59 (m, 1H, H-2′), 4.50 (m, 1H, H-5′), 4.48 (m, 1H, H-6′), 4.06 (s, 1H, H-19), 3.51 (m, 1H, H-3), 1.99 (m, 1H, H-16), 1.86 (m, 1H, H-18), 1.84 (m, 1H, H-2), 1.70-0.70 (21H), 1.16 (s, 3H), 1.02 (s, 3H), 0.92 (s, 3H), 0.84 (s, 3H), 0.83 (s, 3H), 0.77 (s, 3H), 0.73 (s, 3H); ¹³C NMR (Pyr-d5): 179.99, 98.05, 86.23, 81.72, 76.44, 73.67, 73.42, 69.62, 63.84, 56.29, 51.66, 47.17, 46.59, 41.07, 40.54, 39.15, 38.87, 37.65, 36.84, 34.32, 34.11, 33.12, 32.43, 29.23, 29.18, 28.65, 26.95, 26.36, 24.04, 22.50, 21.51, 18.71, 17.12, 17.02, 15.85, 14.13; HR-ESI-MS m/z 641.4043 [M+Na]⁺ (calculated for C₃₆H₅₈O₈Na, 641.4029).

3-O-β-D-Xylopyranoside of 28-Oxyallobetulin (Compound 44)

This compound was prepared from the acceptor 32 (100 mg, 0.22 mmol), and the donor 54 (200 mg, 0.33 mmol) in the same manner as that described for compound 9. Purification by flash chromatography (CH₂Cl₂:CH₃OH, 49:1 to 47:3) afforded 44 as a white solid (28 mg, 22%, 2 steps). I.R.: 3230, 2922, 2853, 1757, 1443, 1386, 1260, 1166, 1044, 966, 921, 712; ¹H NMR (Pyr-d5): 4.88 (d, 1H, J=7.40 Hz, H-1′), 4.43 (m, 1H, H-5′), 4.28 (m, 1H, H-4′), 4.22 (m, 1H, H-3′), 4.07 (m, 1H, H-2′), 4.06 (s, 1H, H-19), 3.82 (m, 1H, H-5′), 3.37 (m, 1H, H-3), 2.24 (m, 1H, H-2), 1.95 (m, 1H, H-2), 1.80-0.70 (24H), 1.32 (s, 3H), 1.03 (s, 3H), 1.00 (s, 3H), 0.93 (s, 3H), 0.89 (s, 3H), 0.79 (s, 3H), 0.78 (s, 3H); ¹³C NMR (Pyr-d5): 179.95, 108.16, 88.94, 86.24, 79.08, 75.98, 71.65, 67.57, 56.37, 51.76, 47.18, 46.58, 41.10, 40.55, 40.10, 39.52, 37.53, 36.85, 34.40, 34.10, 33.11, 32.44, 29.18, 28.65, 28.41, 27.32, 26.96, 26.38, 24.05, 21.53, 18.67, 17.13, 17.11, 15.86, 14.08; HR-ESI-MS m/z 611.3914 [M+Na]⁺ (calculated for C₃₅H₅₆O₇Na, 611.3924).

1,2,3,4,6-Penta-O-benzoyl-α,β-D-plucopyranose (Compound 45)

BzCl (77 mL, 666 mmol) was slowly added to a cooled solution (ice-water bath) of D-glucose (20.0 g, 111 mmol) in anhydrous pyridine (280 mL) with DMAP (136 mg, 1.1 mmol) as catalyst. The reaction was performed overnight at room temperature with constant stirring and then quenched with CH₃OH (31 mL). The mixture was diluted with CH₂Cl₂ and washed with cold H₂SO₄ 3N, saturated NaHCO₃ solution and brine. The solvents of the dried solution (MgSO₄) were evaporated under reduced pressure and the residue was purified by flash chromatography (CH₂Cl₂) to give 45 as a white solid (71.6 g, 92%): R_{f} 0.68 (CH₂Cl₂); mp 172-174° C.; [α]²⁰_{D}+104.9° (c 1.25, CHCl₃). ¹H and ¹³C NMR spectral data of 45 were in agreement with those published in the literature (Trujillo, M. et al., J. Org. Chem. 1994, 59, 6637-6642; D'Accorso, N. B. et al., Carbohyd. Res. 1983, 124, 177-184): HR-ESI-MS m/z 723.1818 [M+Na]⁺ (calculated for C₄₁H₃₂O₁₁Na, 723.1842).

2,3,4,6-Tetra-O-benzoyl-α,β-D-glucopyranose (Compound 46)

HBr/HOAc (10 mL, 33%) was added under N₂ to a solution of 45 (10.0 g, 14.3 mmol) in dry CH₂Cl₂ (42 mL). The reaction mixture was stirred at room temperature for 4 h, then, the solution was washed with saturated NaHCO₃ solution and brine. The organic layer was dried (MgSO₄), filtered and the solvents were evaporated under reduced pressure. After the residue was dissolved in acetone (75 mL) and water (3 mL), Ag₂CO₃ (6.50 g, 23.6 mmol) was added portion wise. The hydrolysis was performed 1 h at room temperature with constant stirring, then, the mixture was filtered through a bed of Celite. The filtrate was concentrated under reduced pressure and the residue was purified by flash chromatography (CH₂Cl₂:CH₃OH 99:1 to 49:1) to give 46 as a white foam (7.32 g, 86%): R_{f} 0.28 (CH₂Cl₂:CH₃OH 99:1); mp 116-118° C., lit.⁵⁶ mp 118-120° C.; [α]²⁰_{D} +70.1° (c 1.42, CHCl₃), lit.⁵⁶ [α]²²_{D} +72.2° (c 0.5, CHCl₃). ¹H and ¹³C NMR spectral data of 46 were in agreement with those published in the literature (Fukase, K. et al., Chem. Express 1993, 8, 409-412; Salinas, A. E. et al., Carbohyd. Res. 1987, 170, 71-99): HR-ESI-MS m/z 619.1567 [M+Na]⁺ (calculated for C₃₄H₂₈O₁₀Na, 619.1580).

2,3,4,6-Tetra-O-Benzoyl-α,β-D-Glucopyranose Trichloroacetimidate (Compound 47)

CCl₃CN (6 mL, 59.8 mmol) was added to a solution of 46 (5.81 g, 9.74 mmol) and Cs₂CO₃ (315 mg, 0.97 mmol) in CH₂Cl₂ (100 mL). The reaction was stirred 4 h at room temperature and then filtered off. The solvents of the filtrate were evaporated under reduced pressure and the residue was purified by flash chromatography (CH₂Cl₂) to give 47 as a white crystalline powder (6.13 g, 85%): R_{f} 0.64 (CH₂Cl₂:CH₃OH 99:1); [α]²⁰_{D} +76.5° (c 1.67, CHCl₃). ¹H and ¹³C NMR spectra data of 26 were in agreement with those published in the literature (Fukase, K., supra). HR-ESI-MS m/z 778.0410 [M+K]⁺ (calculated for C₃₆H₂₈NO₁₀Cl₃K, 778.0415).

1,2,3,4-Tetra-O-Benzoyl-α,β-L-Rhamnopyranose (Compound 48)

This compound was prepared from L-rhamnose (2.05 g, 12.5 mmol) in the same manner as that described for compound 45. Purification by flash chromatography (CH₂Cl₂) afforded 48 as a white crystalline powder (5.95 g, 82%): R_{f} 0.65 (CH₂Cl₂); [α]²⁰_{D} +33.6° (c 0.25, CHCl₃). ¹H NMR (CDCl₃) δ: 1.52 (d, 3H, J=6.2 Hz, H-6), 4.20 (m, 1H, H-5), 5.85 (t, 1H, J=9.6 Hz, H-4), 5.91 (dd, 1H, J=10.0 Hz, J=3.2 Hz, H-3), 6.24 (d, 1H, J=3.0 Hz, H-2), 6.54 (brs, 1H, H-1), 7.20-7.25 (m, 2H, H—Ar), 7.28-7.41 (m, 5H, H—Ar), 7.44-7.54 (m, 4H, H—Ar), 7.58-7.64 (m, 1H, H—Ar), 7.88-7.92 (m, 2H, H—Ar), 7.97-8.05 (m, 4H, H—Ar), 8.23-8.27 (m, 2H, H—Ar). ¹³C NMR (CDCl₃) δ: 17.84 (C-6), 69.88 (C-5), 71.44 (C-2), 71.62 (C-3), 71.75 (C-4), 91.38 (C-1), 128.39-133.75 (C—Ar), 164.27, 165.51, 165.74, 165.85 (4×CO). HR-ESI-MS m/z 603.1613 [M+Na]⁺ (calculated for C₃₄H₂₈O₉Na, 603.1631).

2,3,4-Tri-O-benzoyl-α,β-L-rhamnopyranose trichloroacetimidate (Compound 49)

HBr/HOAc (2.3 mL, 33%) was added at room temperature under N₂ to a solution of 48 (2.31 g, 3.98 mmol) in dry CH₂Cl₂ (10 mL). The reaction mixture was stirred at room temperature for 2 h, then, the solution was washed with saturated NaHCO₃ solution and brine. The organic layer was dried over MgSO₄, filtered and the solvents were evaporated under reduced pressure. After the residue was dissolved in acetone (19 mL) and water (0.8 mL), Ag₂CO₃ (1.50 g, 5.44 mmol) was added portion wise. The hydrolysis was performed 1 h at room temperature with constant stirring, then, the mixture was filtered through a bed of Celite. The filtrate was concentrated under reduced pressure and dissolved in CH₂Cl₂ (50 mL). Cs₂CO₃ (130 mg, 0.40 mmol) was added, followed by CCl₃CN (2.4 mL, 23.9 mmol) and the reaction was stirred 4 h at room temperature. The mixture was then filtered off, concentrated under reduced pressure and the residue was purified by flash chromatography (CH₂Cl₂) to give 49 as a white crystalline powder (1.78 g, 72%, 2 steps): R_{f} 0.74 (CH₂Cl₂); [α]²⁰_{D}+83.6° (c 1.33, CHCl₃), lit.⁴² [α]²⁰_{D} +97.5° (c 1.0, CHCl₃). ¹H and ¹³C NMR spectra data of 49 were in agreement with those published in the literature (Ziegler, T. et al., Tetrahedron: Asymmetry 1998, 9, 765-780). HR-ESI-MS m/z 658.0189 [M+Kr]⁺ (calculated for C₂₉H₂₄NO₈Cl₃K, 658.0204).

1,2,3,4-Tetra-O-Benzoyl-α,β-D-Arabinopyranose (Compound 50)

This compound was prepared from D-arabinose (4.92 g, 32.8 mmol) in the same manner as that described for compound 45. Purification by flash chromatography (CH₂Cl₂) afforded 50 as a white crystalline powder (16.5 g, 89%): R_{f} 0.59 (CH₂Cl₂); [α]²⁰_{D}-274.2° (c 1.00, CHCl₃). ¹H NMR (CDCl₃) δ: 4.21 (dd, 1H, J=13.4 Hz, J=1.8 Hz, H-5a), 4.44 (d, 1H, J=13.0 Hz, H-5β), 5.93 (s, 1H, H-4), 6.10 (brs, 2H, H-2, H-3), 6.90 (brs, 1H, H-1), 7.26-7.34 (m, 4H, H—Ar), 7.42-7.56 (m, 6H, H—Ar), 7.61-7.68 (m, 2H, H—Ar), 7.88-7.93 (m, 4H, H—Ar), 8.13-8.18 (m, 4H, H—Ar). ¹³C NMR (CDCl₃) δ: 63.07 (C-5), 67.82 (C-2), 68.23 (C-3), 69.53 (C-4), 91.12 (C-1), 128.44-133.89 (C—Ar), 164.73, 165.62, 165.76, 165.79 (4×CO). HR-ESI-MS m/z 589.1457 [M+Na]⁺ (calculated for C₃₃H₂₆O₉Na, 589.474).

2,3,4-Tri-O-Benzoyl-α,β-D-Arabinopyranose Trichloroacetimidate (Compound 51)

This compound was prepared from 50 (5.70 g, 10.1 mmol) in the same manner as that described for compound 49. Purification by flash chromatography (CH₂Cl₂) afforded 51 as a white foam (4.76 g, 78%, 2 steps): R_{f} 0.55 (CH₂Cl₂); [α]²⁰_{D}-182.8° (c 1.00, CHCl₃). ¹H NMR (CDCl₃) δ: 4.19 (dd, 1H, J=13.3 Hz, J=2.0 Hz, H-5α), 4.43 (d, 1H, J=12.8 Hz, H-5β), 5.88 (m, 1H, H-4), 6.02 (ddd, 2H, J=16.7 Hz, J=10.7 Hz, J=3.0 Hz, H-2, H-3), 6.83 (d, 1H, J=3.0 Hz, H-1), 7.26-7.33 (m, 2H, H—Ar), 7.34-7.40 (m, 2H, H—Ar), 7.44-7.55 (m, 4H, H—Ar), 7.60-7.66 (m, 1H, H—Ar), 7.84-7.88 (m, 2H, H—Ar), 7.96-8.00 (m, 2H, H—Ar), 8.09-8.15 (m, 2H, H—Ar), 8.64 (brs, 1H, NH). ¹³C NMR (CDCl₃) δ: 63.18 (C-5), 68.00 (d, C-2, C-3), 69.45 (C-4), 90.89 (CCl₃), 94.35 (C-1), 128.38-133.57 (C—Ar), 160.80 (C═NH), 165.59, 165.66, 165.69 (3×CO). HR-ESI-MS m/z 644.0076 [M+K]⁺ (calculated for C₂₈H₂₂NO₈Cl₃K, 644.0048).

2,3,4,6-Tetra-O-Benzoyl-α,β-D-Galactopyranose Trichloroacetimidate (Compound 52)

This compound was prepared according to Rio et al. procedure (Rio, S. et al. Carbohydr. Res. 1991, 219, 71-90) from D-galactose. ¹H and ¹³C NMR spectra data of 52 were in agreement with those published in the literature (Rio, S., supra).

2,3,4,6-Tetra-O-Benzoyl-α,β-D-Mannopyranose Trichloroacetimidate (Compound 53)

This compound was prepared according to Ikeda et al. procedure (Ikeda, T. et al. Bioorg. Med. Chem. Lett. 1997, 7, 2485-2490) from D-mannose. ¹H and ¹³C NMR spectra data of 53 were in agreement with those published in the literature (Ikeda, T., supra).

2,3,4-Tri-O-Benzoyl-α,β-D-Xylopyranose Trichloroacetimidate (Compound 54)

This compound was prepared according to Schmidt et al. procedure (Schmidt, R. R. et al. Trichloroacetimidates. In: Carbohydrates in Chemistry and Biology, Part I: Chemistry of Saccharides, Wiley-VCH, Weinheim, 2000, Vol 1, pp. 5-59) from D-xylose. ¹H and ¹³C NMR spectra data of 54 were in agreement with those published in the literature (Chen, L. et al. Carbohydr. Res. 2002, 337, 2335-2341).

Cell Lines and Culture Conditions

Human lung carcinoma (A-549), human colon adenocarcinoma (DLD-1), human normal fibroblasts (WS1), mice melanoma (B16-F1), Human glioma (U-251), Human hepatocellular carcinoma (HEP G2), Human prostate adenocarcinoma (PC-3), Human ovary teratocarcinoma metastatic (PA-1), Human breast adenocarcinona metastatic (MDA-MB-231), Human breast adenocarcinoma (MCF-7) and Human malignant melanoma (SK-MEL-2) cell lines were obtained from the American Type Culture Collection (ATCC). All cell lines were cultured in minimum essential medium containing Earle's salts and L-glutamine (Mediatech Cellgro, Va.), to which was added 10% fetal bovine serum (Hyclone), vitamins (1×), penicillin (100 I.U./mL) and streptomycin (100 μg/mL), essential amino acids (1×) and sodium pyruvate (1×) (Mediatech Cellgro, Va.). Cells were kept at 37° C. in a humidified environment containing 5% CO₂.

Cytotoxicity Assay

Exponentially growing cells were plated in 96-well microplates (Costar, Corning Inc.) at a density of 5×10³ cells per well in 100 μL of culture medium and were allowed to adhere for 16 hours before treatment. Increasing concentrations of each compound in DMSO (Sigma-Aldrich) were then added (100 μL per well) and the cells were incubated for 48 h. The final concentration of DMSO in the culture medium was maintained at 0.5% (volume/volume) to avoid solvent toxicity. Cytotoxicity was assessed using resazurin (O'Brien, J. et al., Eur. J. Biochem. 2000, 267, 5421-5426) on an automated 96-well Fluoroskan Ascent F1™ plate reader (Labsystems) using excitation and emission wavelengths of 530 nm and 590 nm, respectively. Fluorescence was proportional to the cellular metabolic activity in each well. Survival percentage was defined as the fluorescence in experimental wells compared to that in control wells after subtraction of blank values. Each experiment was carried out three times in triplicata. IC₅₀ results were expressed as mean±standard deviation.

### Example 2

### Extraction and Synthesis of Triterpenes and Triterpene Derivatives

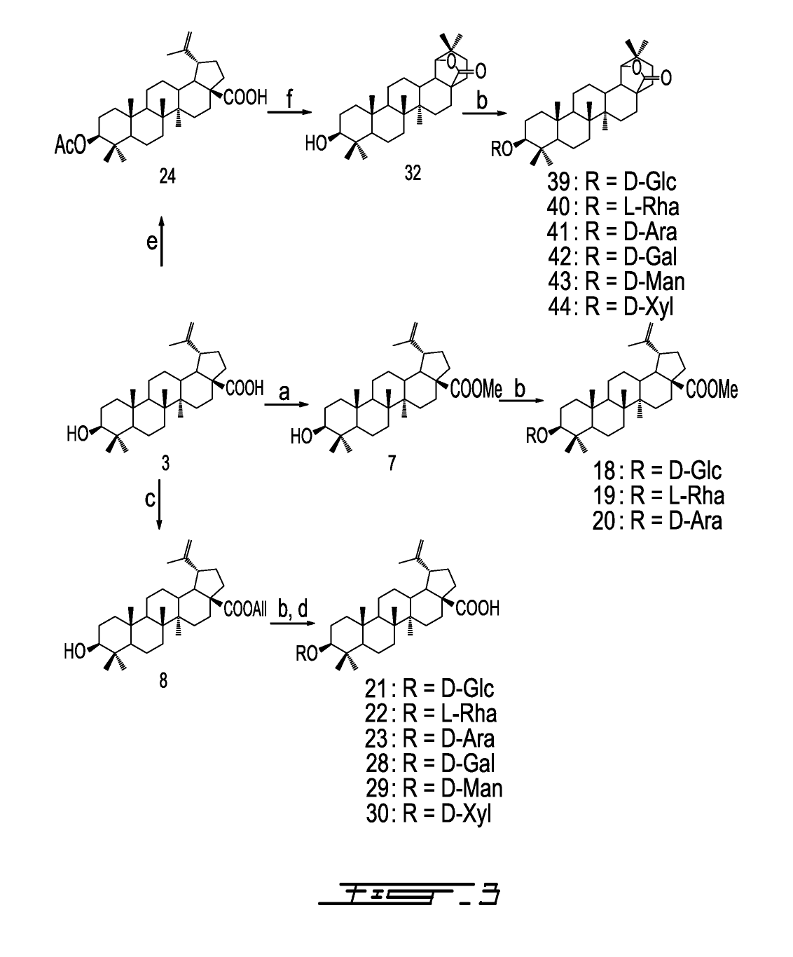

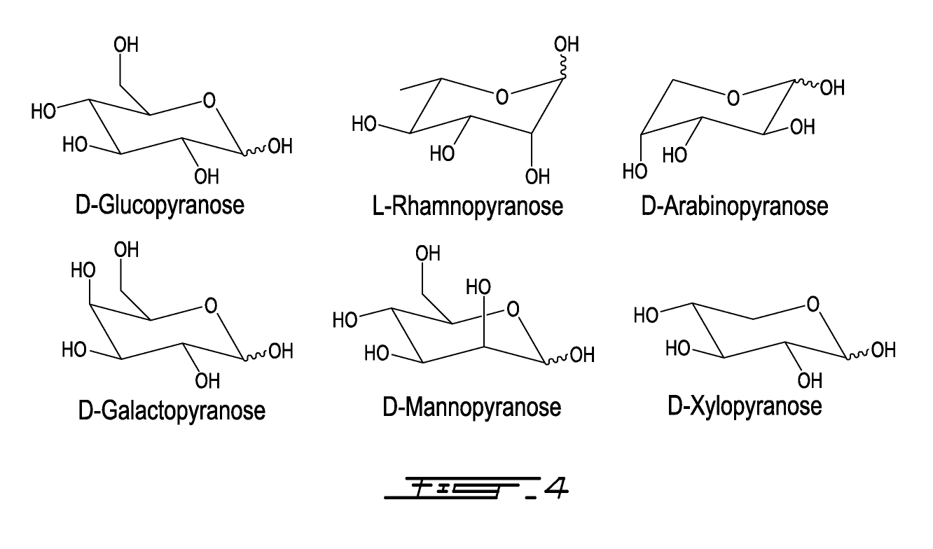

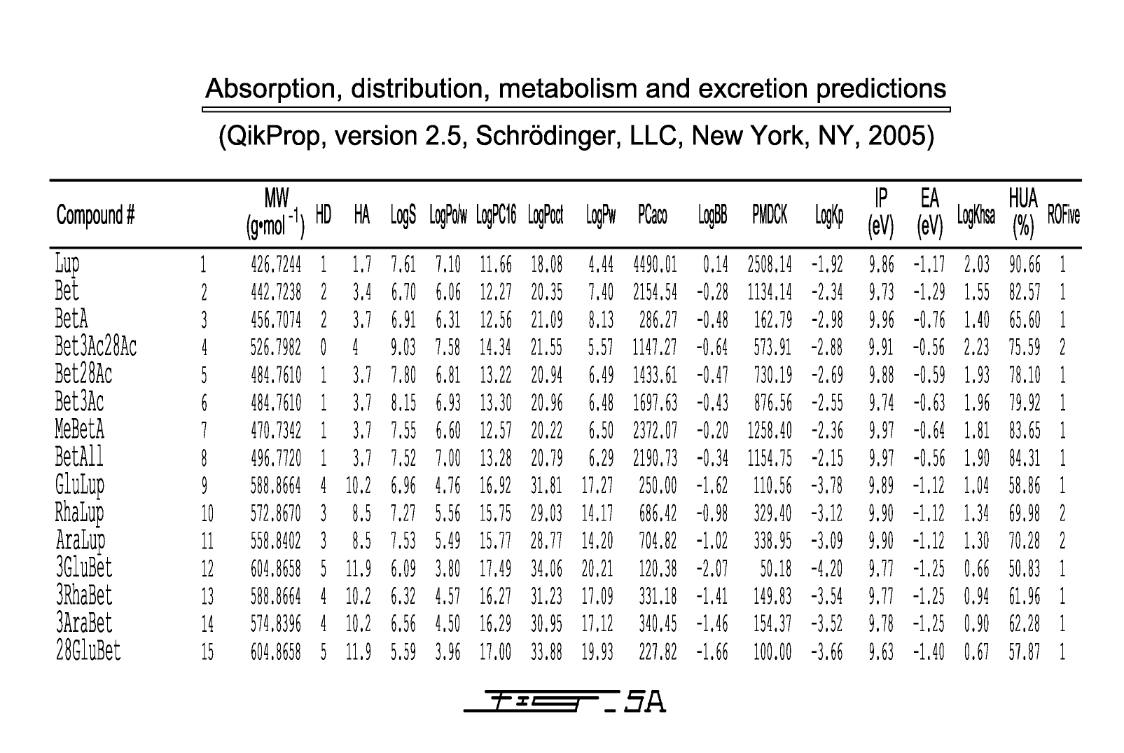

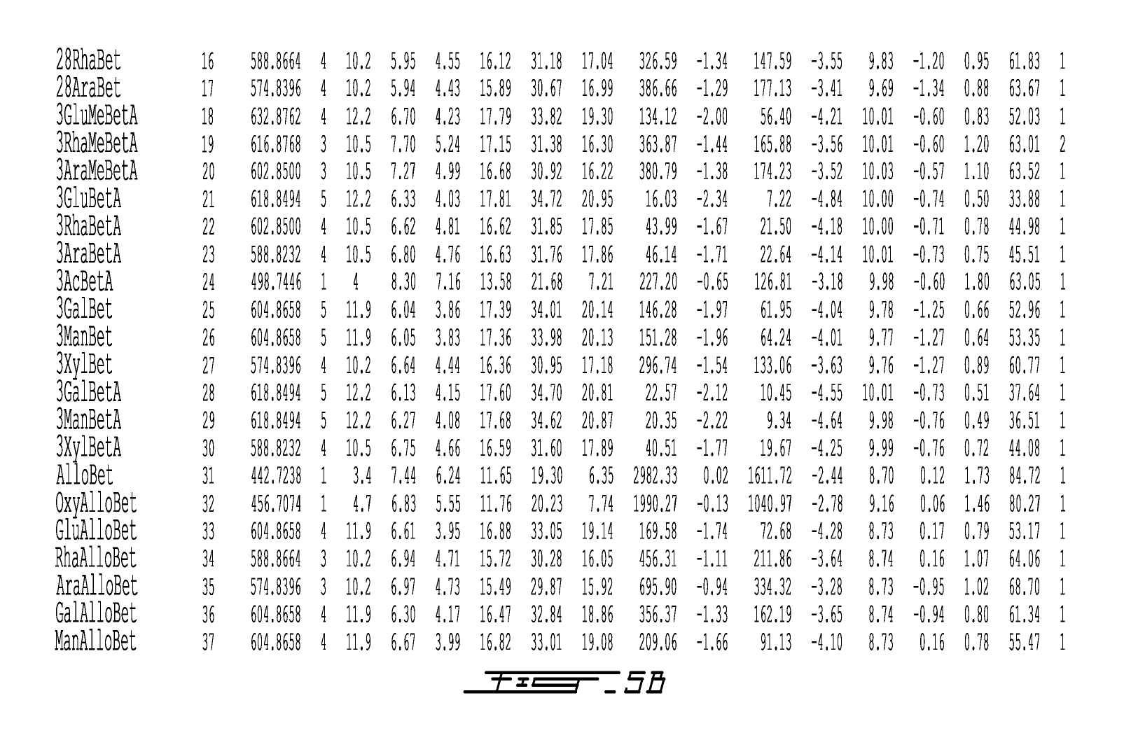

The external bark of yellow and white birches were first refluxed in CHCl₃. Purification of the extracts on silica gel followed by treatment with activated charcoal gave, respectively, the natural triterpenes 1 (1.2%) and 2 (17%). To perform the glycosidation at the C-3 and C-28 positions of 2, the corresponding acetates were prepared. As the reactivity of the C-28 hydroxyl group of 2 is much higher than the one at C-3,28-acetoxybetulin (5) was obtained in moderate yield (73%) by using an excess of acetic anhydride (Ac₂O) in CH₂Cl₂ during a 24 h period at room temperature. As shown in FIG. 2, diacetylation of 2 with Ac₂O, pyridine and a catalytic amount of dimethylaminopyridine (DMAP) in CH₂Cl₂ afforded 3,28-diacetoxybetulin (4) in excellent yield (95%) (Hiroya, K. et al., Bioorg. Med. Chem. 2002, 10, 3229-3236). Subsequent selective deprotection of the C-28 alcohol using Mg(OCH₃)₂ in dry CH₃OH and THF furnished the 3-acetoxybetulin (6) in good yield (85%) as previously reported (Xu, Y.-C. et al., C. J. Org. Chem. 1996, 61, 9086-9089). However, it is important to note that, in the same experimental conditions, contrary to the results of Xu and co-workers, the reaction was complete after 4 h instead of 3 days. As shown in FIG. 3, the methyl ester 7 of the commercially available 3 was synthesized in moderate yield (71%) by treatment with iodomethane in the presence of DBU (Mal, D. Synth. Commun. 1986, 16, 331-335). Methods used to regenerate the carboxylic acid (NaOH 1N refluxed in DMF or dioxane and Ba(OH)₂.8H₂O in CH₃OH) from methyl betulinate glycosides (18, 19, 20) failed to yield the corresponding betulinic acid glycosides (21, 22, 23). Therefore, another more versatile protection group for the C-28 acid function was considered. To this end, the synthesis of allyl betulinate (8) was carried out in good yield (84%) by reaction of 3 using allyl bromide in DMF in the presence of K₂CO₃ (Plé, K. et al., Eur. J. Org. Chem. 2004, 1588-1603). Allobetulin (31) was easily obtained from the well known Wagner-Meerwein rearrangement by the action of Fe(NO₃)₃/SiO₂ (1/4) on betulin (2) in refluxed CH₂Cl₂. 28-oxyallobetulin (32) was equally obtained from the Wagner-Meerwein rearrangement by the action of FeCl₃/SiO₂ (1/4) on 3-acetoxybetulinic acid (24) in refluxed CH₂Cl₂.

### Example 3

### Synthesis of Activated Sugars

Protection of sugar alcohols (FIG. 3) was achieved by using benzoyl chloride in pyridine with DMAP as catalyst to afford 1,2,3,4,6-penta-O-benzoyl-α,β-D-glucopyranose (24, 92%), 1,2,3,4-tetra-O-benzoyl-α,β-L-rhamnopyranose (27, 82%) and 1,2,3,4-tetra-O-benzoyl-α,β-D-arabinopyranose (29, 89%) (Trujillo, M. et al., J. Org. Chem. 1994, 59, 6637-6642). Thereafter, bromination (HBr—HOAc 33%) of the benzoylated sugars followed by basic hydrolysis with silver carbonate (Ag₂CO₃) in acetone:H₂O 20:1 allowed the selective deprotection of the anomeric position in good yield for 2,3,4,6-tetra-O-benzoyl-α,β-D-glucopyranose (25, 86%) and in a quantitative way for L-rhamnose and D-arabinose derivatives (Deng, S et al., J. Org. Chem. 1999, 64, 7265-7266). Finally, trichloroacetimidate derivatives 26 (85%) (Fukase, K et al., Chem. Express 1993, 8, 409-412), 28 (72%, 2 steps) (Ziegler, T. et al., Tetrahedron: Asymmetry 1998, 9, 765-780), 30 (78%, 2 steps) were synthesized from the corresponding 1-OH sugars according to Schmidt's procedure (Schmidt, R. R. Adv. Carbohydr. Chem. Biochem. 1994, 50, 21-123) using trichloroacetonitrile (CCl₃CN) and a catalytic amount of cesium carbonate (Cs₂CO₃) in CH₂Cl₂ (Urban, F. J. et al., Tetrahedron Lett. 1990, 31, 4421-4424).

### Example 4

### Synthesis of Glycosides

Glycosidations of the lupane- and germanicane-type triterpenoids were achieved by the reaction of acceptors (1, 5, 6, 7, 8, 31, 32) with donors (47, 49, 51-54) at room temperature in CH₂Cl₂ under the catalytic promotion of the Lewis acid trimethylsilyl trifluoromethanesulfonate (TMSOTf) (Deng, S. et al., J. Org. Chem. 1999, 64, 7265-7266). Subsequent removal of the protecting groups (benzoyl and acetate) by using NaOH 0.25 N in CH₃OH:THF:H₂O 1:2:1 gave glycosides (9-23, 25-30, 33-44). Betulinic acid glycosides (21-23, 28-30) were only obtained after the regeneration of the C-28 acid function in the presence of a catalytic amount of tetrakistriphenylphosphine palladium Pd⁰(PPh₃)₄ and pyrrolidine in dry THF (Plé, K. et al., Eur. J. Org. Chem. 2004, 1588-1603). Since the glycosyl donors contained benzoyl participating neighboring groups, exclusively 1,2-trans-glycosides were synthesized as confirmed by ¹H NMR experiments.

### Example 5

### Solubility and Pharmacological Properties of Triterpenes and Glycosides Derivatives

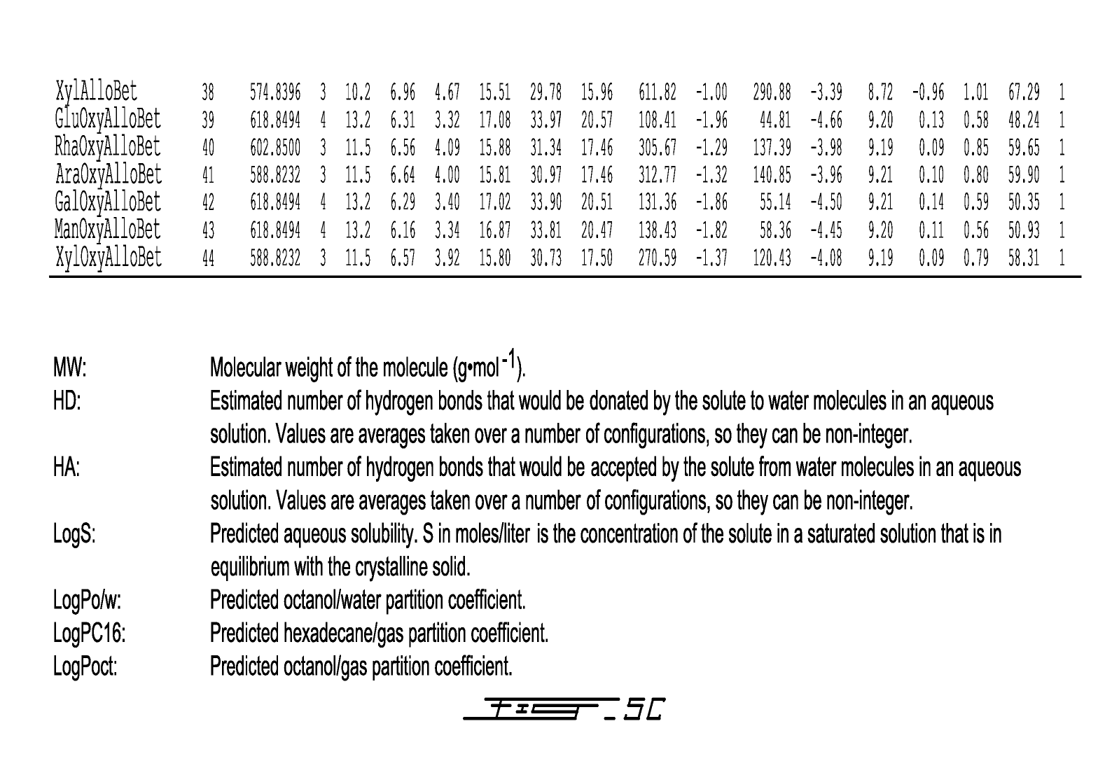

Each compound (10 mg) was dissolved in 0.5 mL of each solvent and the resulting solution was ultrasonicated. Then, the solution was qualitatively characterized according to the solubility: homogeneous solution (+), heterogeneous solution (±), precipitated solution (−). The glycosides showed a greater solubility than corresponding triterpenes in the polar solvents (DMSO and CH₃OH) used for bioassays (Table 1 below). FIG. 5 provides the predicted absorption, distribution, metabolism and excretion of the different triterpenes and triterpene derivatives.

TABLE 1
Solubility of glycosides and corresponding triterpenes

| | | Solubility^{a} | | |
|---|---|---|---|---|
| Compound | | CH₂Cl₂ | DMSO | CH₃OH |
| 1 | Lup | + | − | − |
| 2 | Bet | ± | ± | − |
| 3 | BetA | ± | + | − |
| 4 | BetDiAc | + | − | − |
| 5 | Bet28Ac | + | − | − |
| 6 | Bet3Ac | + | − | − |
| 7 | MeBetA | + | − | − |
| 8 | BetAll | + | − | − |
| 9 | GluLup | + | + | ± |
| 10 | RhaLup | + | + | ± |
| 11 | AraLup | + | + | ± |
| 12 | 3GluBet | − | + | + |
| 13 | 3RhaBet | − | + | + |
| 14 | 3AraBet | − | + | + |
| 15 | 28GluBet | − | + | ± |
| 16 | 28RhaBet | − | + | ± |
| 17 | 28AraBet | − | + | ± |
| 18 | GluMeBetA | + | + | ± |
| 19 | RhaMeBetA | + | + | ± |
| 20 | AraMeBetA | + | + | ± |
| 21 | GluBetA | − | + | + |
| 22 | RhaBetA | − | + | + |
| 23 | AraBetA | − | + | + |

^{a}+: soluble, ±: not very soluble, −: insoluble

### Example 6

### Cytotoxic Activity Against A-549, DLD-1 and B16-F1

The cytotoxicity of triterpenes (1-8) and corresponding glycosides (9-30) (Table 2 below) as well as of germanicane-type triterpenes and glycosides (31-44) (Table 3 below) was assessed towards human cancer (A-549, DLD-1), mouse melanoma (B16-F1) and human normal skin fibroblast (WS1) cell lines using the resazurin reduction test (RTT test) as previously described (O'Brien, J. et al., Eur. J. Biochem. 2000, 267, 5421-5426). Measurements of fluorescence were carried out after 48 continuous hours of contact between compounds and cells. Results presented in Tables 2 and 3 below express the concentration inhibiting 50% of the cell growth (IC₅₀). Known for its activity against A-549, betulinic acid (3) was used as a positive control in this experimentation. Based on the IC₅₀ values, compounds with IC₅₀<20 μM were considered strongly active, those with IC₅₀ ranging from ˜20 to 75 μM were considered moderately active and those with IC₅₀ ranging from ˜75 to 165 μM were considered weakly active. Otherwise, the compounds were considered to be inactive. The cytotoxic activity of some of these compounds was also assessed using the Hoechst DNA assay (Table 4 below).

TABLE 2
In vitro cytotoxicity of lupane-type triterpenoids and glycosides,
as measured by the resazurin metabolism assay, O'Brien, J. et al., Eur. J. Biochem.
2000, 267, 5421-5426.

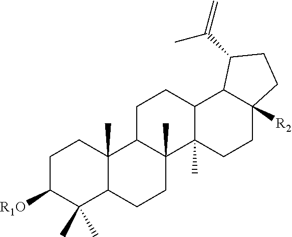

| | | | Cell Line IC₅₀ (μM ± SD)^{a} | | | |
|---|---|---|---|---|---|---|
| Compound | R₁ | R₂ | A-549^{b} | DLD-1^{c} | B16-F1^{d} | WS-1^{e} |
| 1 | H | CH₃ | 165 ± 8 | 125 ± 6 | 104 ± 6 | 63 ± 3 |
| 2 | H | CH₂OH | 3.80 ± 0.09 | 6.6 ± 0.3 | 13.8 ± 0.5 | 3.58 ± 0.07 |
| 3 | H | COOH | 10.3 ± 0.4 | 15.0 ± 0.3 | 16.1 ± 0.5 | 12 ± 1 |
| 4 | Ac | CH₂OAc | >95 | >95 | >95 | >95 |
| 5 | H | CH₂OAc | 75 ± 7 | 56 ± 4 | 43 ± 2 | 44 ± 2 |
| 6 | Ac | CH₂OH | >253 | >253 | >253 | >253 |
| 24 | Ac | COOH | 18 ± 2 | 20 ± 2 | nd | 57 ± 6 |
| 7 | H | COOCH₃ | 19 ± 3 | 25 ± 4 | 26 ± 1 | 19 ± 2 |
| 8 | H | COOAll | >225 | >225 | >225 | >225 |
| 9 | Glc | CH₃ | 14 ± 1 | 14 ± 1 | 15.0 ± 0.7 | 13.3 ± 0.5 |
| 10 | Rha | CH₃ | >178 | >178 | >178 | >178 |
| 11 | Ara | CH₃ | 28 ± 2 | 50 ± 6 | 27 ± 2 | 15.8 ± 0.8 |
| 12 | Glc | CH₂OH | >200 | >200 | >200 | >200 |
| 13 | Rha | CH₂OH | 22 ± 3 | 50 ± 10 | 18 ± 1 | 33 ± 5 |
| 14 | Ara | CH₂OH | 41 ± 3 | 63 ± 8 | 38 ± 3 | 59 ± 5 |
| 25 | Gal | CH₂OH | >100 | >100 | nd | >100 |
| 26 | Man | CH₂OH | 7.5 ± 0.1 | 11.0 ± 0.5 | nd | 5.3 ± 0.5 |
| 27 | Xyl | CH₂OH | 90 ± 10 | >100 | nd | >100 |
| 15 | H | CH₂O-Glc | >248 | >248 | >248 | >248 |
| 16 | H | CH₂O-Rha | >228 | >228 | >228 | >228 |
| 17 | H | CH₂O-Ara | >175 | >175 | >175 | >175 |
| 18 | Glc | COOCH₃ | 8.4 ± 0.3 | 3.93 ± 0.09 | 7.1 ± 0.3 | 9.3 ± 0.2 |
| 19 | Rha | COOCH₃ | 59 ± 3 | >183 | 55 ± 2 | 53 ± 2 |
| 20 | Ara | COOCH₃ | 13.5 ± 0.6 | 18 ± 1 | 13.3 ± 0.4 | 12.5 ± 0.4 |
| 21 | Glc | COOH | >178 | 32 ± 9 | 49 ± 13 | >178 |
| 22 | Rha | COOH | 2.6 ± 0.6 | 3.9 ± 0.4 | 3.9 ± 0.4 | 31 ± 3 |
| 23 | Ara | COOH | 10 ± 2 | 17 ± 3 | 11 ± 1 | 47 ± 5 |
| 28 | Gal | COOH | >100 | >100 | nd | >100 |
| 29 | Man | COOH | 41 ± 4 | 14.9 ± 0.5 | nd | 16 ± 3 |
| 30 | Xyl | COOH | 14 ± 2 | 19.2 ± 0.8 | nd | 21 ± 1 |

^{a}Data represent mean values (±SD) for three independent experiments made in triplicate.
^{b}Human lung carcinoma.
^{c}Human colorectal adenocarcinoma.
^{d}Mouse melanoma.
^{e}Human normal skin fibroblasts.
Glc: β-D-Glucopyranose.
Rha: α-L-Rhamnopyranose.
Ara: α-D-Arabinopyranose.
Gal: β-D-Galactopyranose.
Man: α-D-Mannopyranose.
Xyl: β-D-Xylopyranose.
Ac: Acetate.
All: Allyl
Nd: not tested.

TABLE 3
In vitro cytotoxicity of germanicane-type triterpenoid saponins:

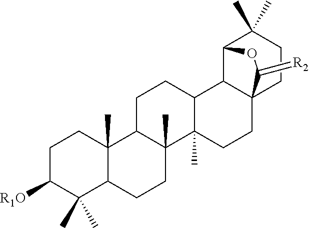

| | | | Cell Line IC₅₀ (μM ± SD)^{a} | | | |
|---|---|---|---|---|---|---|
| Compound | R₁ | R₂ | A-549^{b} | DLD-1^{c} | B16-F1^{d} | WS-1^{e} |
| 31 | H | H₂ | >100 | >100 | nd | >100 |
| 32 | H | O | >100 | >100 | nd | 70 ± 9 |
| 33 | Glc | H₂ | 31 ± 2 | 41.6 ± 0.9 | nd | 45 ± 3 |
| 34 | Rha | H₂ | >100 | >100 | nd | 75 ± 5 |
| 35 | Ara | H₂ | >100 | >100 | nd | >100 |
| 36 | Gal | H₂ | 30 ± 10 | 42 ± 9 | nd | 30 ± 9 |
| 37 | Man | H₂ | >100 | >100 | nd | >100 |
| 38 | Xyl | H₂ | >100 | >100 | nd | >100 |
| 39 | Glc | O | >100 | >100 | nd | >100 |
| 40 | Rha | O | >100 | >100 | nd | >100 |
| 41 | Ara | O | >100 | >100 | nd | >100 |
| 42 | Gal | O | >100 | >100 | nd | >100 |
| 43 | Man | O | >100 | >100 | nd | >100 |
| 44 | Xyl | O | >100 | >100 | nd | >100 |

TABLE 4
In vitro cytotoxicity of lupane-type triterpenoids and
glycosides, as measured by the Hoechst DNA assay:

| | | IC₅₀ ± SD (μM) | | | |
|---|---|---|---|---|---|
| | | Cell lines | | | |
| Compound | | A-549 | DLD-1 | B16-F1 | WS-1_{[CG1]} |
| 1 | Lup | 130 ± 20 | 102 ± 6 | 72 ± 9 | 70 ± 10 |
| 2 | Bet | 4.5 ± 0.3 | 5.9 ± 0.6 | 10.3 ± 0.7 | 5 ± 1 |
| 3 | BetA | 8 ± 1 | 12 ± 1 | 18 ± 2 | 14 ± 2 |
| 4 | BetDiAc | nd | Nd | Nd | nd |
| 5 | Bet28Ac | 49 ± 7 | 46 ± 5 | 35 ± 1 | 47 ± 2 |
| 6 | Bet3Ac | 90 ± 10 | >253 | 42 ± 6 | >180 |
| 7 | MeBetA | 19 ± 2 | 21 ± 1 | 15.7 ± 0.9 | 19 ± 4 |
| 8 | BetAll | >225 | >225 | >225 | >225 |
| 9 | GluLup | 22 ± 2 | 19 ± 1 | 18 ± 2 | 20 ± 2 |
| 10 | RhaLup | >178 | >178 | >178 | >178 |
| 11 | AraLup | 34 ± 2 | 69 ± 7 | 28 ± 1 | 24 ± 1 |
| 12 | 3GluBet | >200 | >200 | >200 | >200 |
| 13 | 3RhaBet | nd | Nd | Nd | nd |
| 14 | 3AraBet | nd | Nd | Nd | nd |
| 15 | 28GluBet | >194 | >194 | >194 | >194 |
| 16 | 28RhaBet | >194 | >194 | >194 | >194 |
| 17 | 28AraBet | >194 | >194 | >194 | >194 |
| 18 | GluMeBetA | 9.3 ± 0.6 | 4.0 ± 0.2 | 7.2 ± 0.8 | 12 ± 2 |
| 19 | RhaMeBetA | 58 ± 2 | >150 | 46 ± 1 | 65 ± 5 |
| 20 | AraMeBetA | 11.7 ± 0.8 | 16.0 ± 0.6 | 12.6 ± 0.5 | 13.2 ± 0.7 |
| 21 | GluBetA | >178 | 12 ± 3 | 17 ± 4 | >178 |
| 22 | RhaBetA | 2.6 ± 0.3 | 3.4 ± 0.5 | 4.2 ± 0.5 | 38 ± 6 |
| 23 | AraBetA | 5.7 ± 0.8 | 10 ± 1 | 10.2 ± 0.6 | 32 ± 2 |

### Example 7

### Cytotoxicity Against Other Cancer Cell Lines

Compounds presented in Table 5 below were also tested in the following tumour cell lines: U-251 (Human glioma), HEP G2 (Human hepatocellular carcinoma), PC-3 (Human prostate adenocarcinoma), PA-1 (Human ovary teratocarcinoma metastatic), MDA-MB-231 (Human breast adenocarcinona metastatic), MCF-7 (Human breast adenocarcinoma) and SK-MEL-2 (Human malignant melanoma).

TABLE 5
In vitro cytotoxicity of selected compounds, as measured by the resazurin metabolism
assay (O'Brien, J. et al., Eur. J. Biochem. 2000, 267, 5421-5426)

| | Cell Line IC₅₀ (μM ± SD)^{a} | | | | | | |
|---|---|---|---|---|---|---|---|
| | | | MDA-MB | | | | |
| Compound | Hep G2^{b} | MCF-7^{c} | 231^{d} | SK-Mel-2^{e} | PA-1^{f} | PC-3^{g} | U-251^{h} |
| 9 | 17.8 ± 0.2 | 16.4 ± 0.5 | 20.9 ± 0.6 | 15.5 ± 0.6 | 13 ± 1 | 30 ± 2 | 17.9 ± 0.7 |
| 11 | 10.0 ± 0.9 | 23 ± 2 | 11 ± 1 | 10.0 ± 0.8 | 9.8 ± 0.6 | 26 ± 3 | 10.1 ± 0.2 |
| 13 | 11.0 ± 0.9 | 19 ± 4 | 33 ± 2 | 110 ± 20 | 180 ± 30 | 61 ± 6 | 170 ± 40 |
| 14 | 38 ± 2 | 61 ± 7 | 49.2 ± 0.9 | 54 ± 3 | 41 ± 5 | 65 ± 6 | 40 ± 7 |
| 18 | 79 ± 5 | 110 ± 6 | 101.7 ± 0.1 | 103 ± 4 | 60 ± 20 | 130 ± 30 | 84 ± 2 |
| 20 | 15 ± 2 | 21 ± 3 | 16.3 ± 0.8 | 16 ± 1 | 16 ± 4 | 17 ± 1 | 15 ± 1 |
| 22 | 20 ± 2 | 16 ± 2 | 19 ± 2 | 20 ± 7 | 8 ± 1 | 20 ± 6 | 20 ± 2 |
| 23 | 66 ± 9 | 45 ± 9 | 57 ± 6 | 62 ± 7 | 20 ± 2 | 110 ± 10 | 70 ± 10 |
| 26 | 8.3 ± 0.4 | 9.2 ± 0.4 | 9.1 ± 0.2 | 8.8 ± 0.4 | 7.6 ± 0.4 | 8.6 ± 0.4 | 8.3 ± 0.4 |
| 29 | 26 ± 2 | 20 ± 2 | 21 ± 2 | 4.7 ± 0.6 | 2.2 ± 0.2 | 27 ± 3 | 6 ± 2 |
| 30 | 43 ± 3 | 23 ± 4 | 40 ± 4 | 36 ± 4 | 7.9 ± 0.9 | 46 ± 8 | 26 ± 6 |
| 33 | 44 ± 5 | 51 ± 2 | 41.2 ± 0.7 | 37 ± 2 | 39 ± 2 | 42 ± 8 | 46 ± 1 |
| 36 | 41 ± 10 | 60 ± 20 | 44 ± 4 | 40 ± 3 | 45 ± 4 | 45 ± 6 | 53 ± 4 |

^{a}Data represent mean values ± standard deviation for three independent experiments made in triplicate.
^{b}Human hepatocellular carcinoma.
^{c}Human breast adenocarcinoma.
^{d}Human breast adenocarcinoma.
^{e}Human melanoma.
^{f}Human ovary teratocarcinoma.
^{g}Human prostate adenocarcinoma.
^{h}Human glioma.

Compounds of the invention are also tested in the following tumour cell lines: Panc 05.04 (Human pancreas adenocarcinoma), K-562 (Human chronic myelogenous leukaemia), A375.S2 (Human skin malignant melanoma), Caco-2 (Human colorectal adenocarcinoma), U-87 (Human colorectal adenocarcinoma) and IMR-90 (Human lung fibroblast).

### Example 8

### In Vivo Antitumoral Evaluation of 3-O-α-L-Rhamnopyranoside Betulinic Acid (22)

Cell lines and mice preparation: The Lewis lung carcinoma cell lines (#CRL-1642, lot # 4372266, ATCC) and the C57BL/6 mouse strain (Charles River Inc., St-Constant, Qc) were used. Cells were grown to 90% confluence in complete DMEM medium containing Earle's salts and L-glutamine (Mediatech Cellgro, Va.), 10% foetal bovine serum (Hyclone), vitamins (1×), penicillin (100 I.U./mL) and streptomycin (100 μg/mL), essential amino acids (1×) and sodium pyruvate (1×) (Mediatech Cellgro, Va.). Cells were then harvested with up and down only. Cells were counted using a hemacytometer and resuspended in DMEM medium without SVF. 100 μL of a solution containing 1×10⁷ cells/mL was inoculated subcutaneously in the right flank of each 6 weeks old mouse on day zero.

Mice were handled and cared for in accordance with the Guide for the Care and Use of Laboratory Animals. Treatment was performed by IP route starting 1 day after tumour injection. Betulinic acid and 3-O-α-L-rhamnopyranoside betulinic acid (22) were dissolved in DMSO and administered at 50, 100 and 200 mg/kg of body weight every 3-4 days. Individual dose were based on the body weight of each mouse. All the mice received a constant injection volume of 100 μL per 25 g of body weight. Control mice were similarly treated IP with the solvent used for the dissolution of drug (DMSO). The experimental mice were weighed daily.

Data analysis: In vivo antitumor activity was evaluated according to the parameters as follows (Miot-Noirault, E. et al. Invest. New Drugs 2004, 22, 369-378):

(a) Calculated tumour weight (CTW): The CTW of each tumour was estimated from two-dimensional measurements performed once a day with a slide calliper, according to the formula: CTW (mg)=(L×W²)/2 with L=length in mm and W=width in mm. Differences in CTW between treated and control groups (DMSO) were analyzed for significance using the U Wilcoxon-Mann-Whitney test and Student t-test. Values of p<0.05 were considered statistically significant.

(b) Treated/Control value (T/C) and Tumour Growth Inhibition (TGI):The T/C was calculated as the ratio of the mean CTW of TW of drug-treated mice versus controls: T/C=(CTW of the drug-treated group on Day X/CTW of the control group on Day X)×100. TGI is 100−(T/C) value.

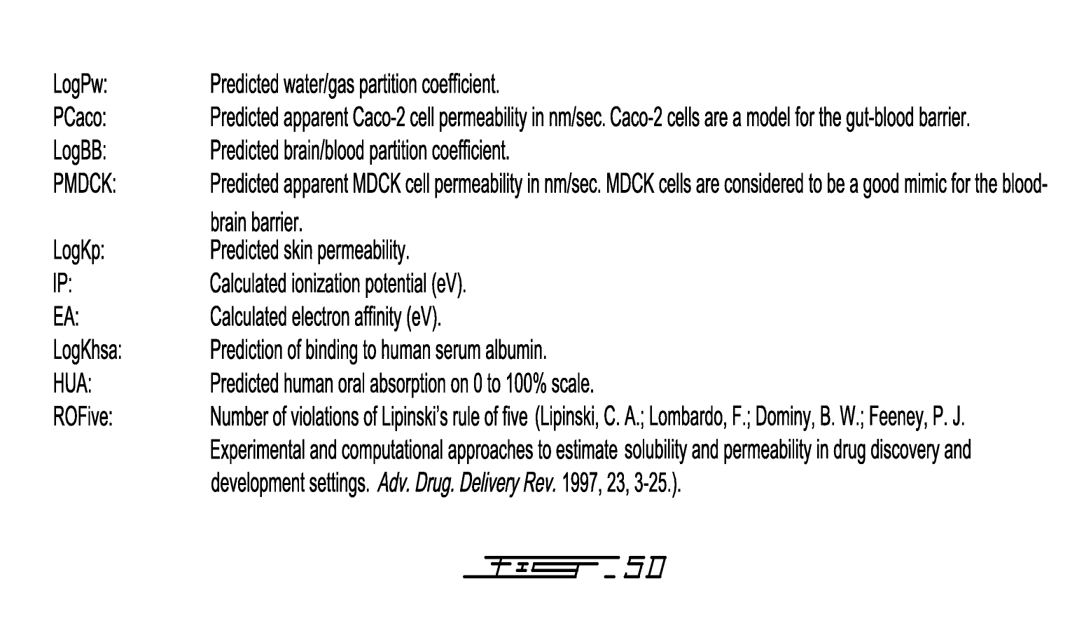

FIG. 6 presents the results of the calculated tumour weight (CTW) on day 11, 12 and 13 for each treatment. Table 6 reports the results of the calculated tumour weight (CTW) and the tumour growth inhibition (TGI) on day 13. The results show that 3-O-α-L-rhamnopyranoside betulinic acid (22) displayed significantly effective tumour growth inhibition (p<0.05) for the doses of 100 (TGI=45%) and 200 (TGI=41%) mg/kg of body weight compared with controls. Moreover, this in vivo antitumoral activity was significantly higher than betulinic acid for the same doses.

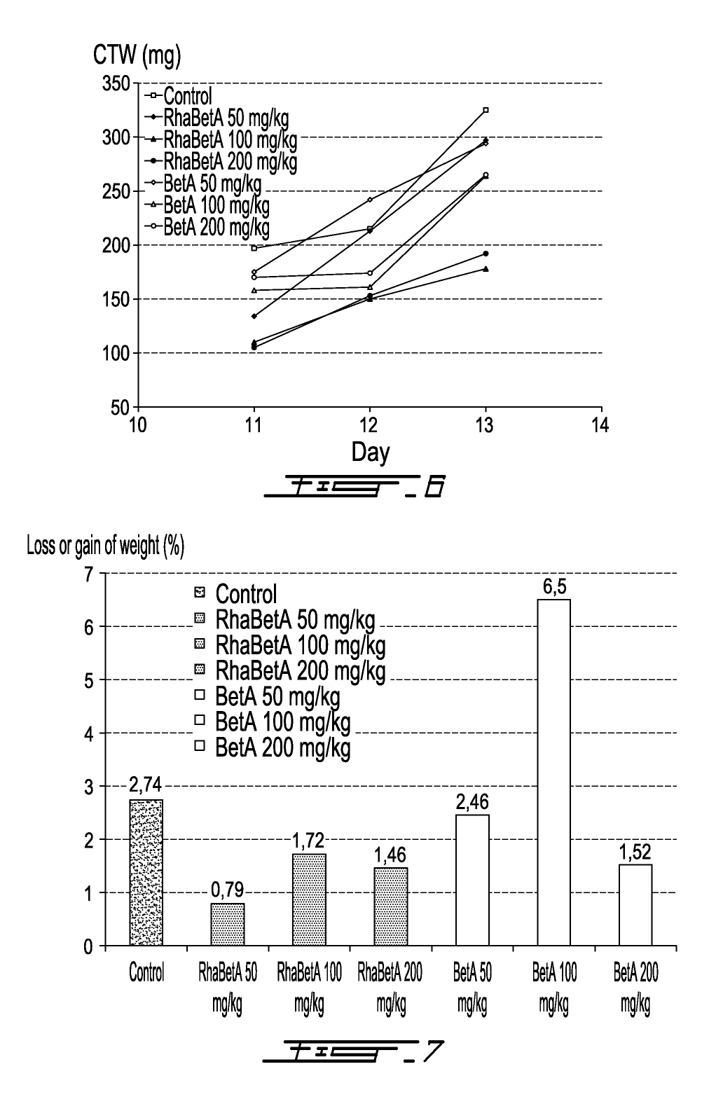

The toxicity of treatment was determined using the body weight of mice. The National Cancer Institute considers that a treatment is toxic if the loss of weight is superior to 20% with regard to the initial weight. FIG. 7 presents the percentage of loss or gain of weight on day 13. It is noteworthy that mice treated with 3-O-α-L-rhamnopyranoside betulinic acid (22) did not show any sign of toxicity or body weight loss compared with controls (FIG. 7).

TABLE 6
Assessment of In vivo antitumoral activity of betulinic
acid (BetA) and 3-O-α-L-rhamnopyranoside betulinic acid
(RhaBetA, 22) against Lewis lung cancer-bearing mice^{a}

| | Number of | Dose | CTW^{b} | T/C^{c} | TGI^{d} |
|---|---|---|---|---|---|
| Drug | animals | (mg/kg) | (mg) | (%) | (%) |
| Control | 10 | — | 325 ± 102 | 100 | — |
| RhaBetA | 10 | 50 | 297 ± 98 | 91 | 9 |
| RhaBetA | 10 | 100 | 178 ± 53^{e} | 55 | 45 |
| RhaBetA | 10 | 200 | 192 ± 50^{e} | 59 | 41 |
| BetA | 10 | 50 | 294 ± 69 | 90 | 10 |
| BetA | 10 | 100 | 264 ± 58 | 81 | 19 |
| BetA | 10 | 200 | 265 ± 58 | 81 | 19 |

^{a}Tumours were measured on day 13 with an electronic calliper
^{b}CTW: Calculated tumour weight
^{c}T/C: Treated/Control (DMSO) × 100%
^{d}TGI: Tumour Growth Inhibition = 100 − T/C (%)
^{e}Significantly different from control (DMSO); Student t-test, p < 0.05; Wilcoxon-Mann-Withney U test, p < 0.05

### Example 9

### Determination of the Maximum Tolerated Dose (MTD) for 3-O-α-L-Rhamnopyranoside Betulinic Acid (22)

Groups of five mice (Charles River) received a single IP injection of 3-O-α-L-rhamnopyranoside betulinic acid (22) in DMSO at doses of 50, 100, 250 and 500 mg/kg of body weight. Individual dose were based on the body weight of each mouse. A group of five control mice received the vehicle (DMSO). All the mice received a constant injection volume of 100 μL per 25 g of body weight. After injection, mice were observed to evaluate general clinical state. For each animal, a score was calculated based on the absence (value 0) or presence (value 1) of diarrhoea, lethargy, rough coat and closed eyes. A clinical state score (CSS) was then calculated per group by summing individual scores. All the mice were weighed daily during 3 days following the injection. The maximal weight loss was determined 24 hours and 3 days following the injection. The MTD was defined as the highest single dose that met all the following criteria: 1) zero death per group; 2) maximal weight loss 20% in non-tumour bearing animals; and 3) CSS value lower than 15.

As shown in Table 7 below, no mortality was obtained and the body weight loss after 24 h (9-14%) was similar for all tested doses. After 3 days, all the mice returned to their initial weight (0%). For groups at 50, 100 and 250 mg/kg of body weight, IP administrations of compound 22 involved no sign of diarrhoea or lethargy. However, at 500 mg/kg of body weight the mice showed signs of diarrhoea and two of them were in lethargy while rough coat and closed eyes were observed in 100% of the mice. Hence, this condition provided the higher CSS (17). According to the criteria defined above, MTD was determined at 250 mg/kg for compound 22.

TABLE 7
Determination of the MTD^{a} for compound 22 after a single IP^{b} injection

| | Number | CSS^{c} | | | | | Max. | |
|---|---|---|---|---|---|---|---|---|
| Dose | of | | | Rough | Closed | | weight | Number |
| (mg/kg) | animals | Diarrhea | Lethargy | coat | eyes | Total | loss^{d} (%) | of deaths |
| Control | 5 | 0 | 0 | 0 | 0 | 0 | 10/0 | 0 |
| 50 | 5 | 0 | 0 | 0 | 0 | 0 | 11/0 | 0 |
| 100 | 5 | 0 | 0 | 0 | 0 | 0 | 14/0 | 0 |
| 250 | 5 | 0 | 0 | 5 | 5 | 10 | 9/0 | 0 |
| 500 | 5 | 5 | 2 | 5 | 5 | 17 | 11/0 | 0 |

^{a}MTD: Maximum tolerated dose
^{b}IP: Intraperitoneal
^{c}CSS: Clinical state score
^{d}Max weight loss after 24 hours and 3 days

This dose can be scaled up to a human equivalent dose (HED) using published conversion tables that take into account the body surface area of the species. The conversion factor from mice to human being 12.3, a MTD of 250 mg/kg for mice is equivalent to 20.33 mg/kg in human. This value (20.33 mg/kg) is divided by a security factor of 10. The calculated MTD is thus 2.33 mg/kg. For an average human weighting 60 kg, the calculated dose is thus 139.8 mg.

### Example 10

### Anti-Inflammatory Activity of Compound 17

Exponentially growing cells were plated in 24-well microplates (BD Falcon) at a density of 2×10⁵ cells per well in 400 μl of culture medium and were allowed to adhere overnight. Cells were then treated or not with positive control N(G)-nitro-L-arginine methyl ester (L-NAME), or increasing concentrations of methanol extracts dissolved in the appropriate solvents, and incubated at 37° C., 5% CO₂ for 24 h. The final concentration of solvent in the culture medium was maintained at 0.5% (volume/volume) to avoid solvent toxicity. Cells were then stimulated with 100 ug/ml lipopolysaccharide (LPS). After 24 h, cell-free supernatants were collected and stored at −80° C. until NO determination using the Griess reaction (Green et al. 1990) with minor modifications. Briefly, 100 μl aliquots of cell supernatants were incubated with 50 μl of 1% sulfanilamide and 50 μl of 0.1% N-1-naphtylethylenediamine dihydrochloride in 2.5% H₃PO₄ at room temperature for 20 min. Absorbance at 540 nm was then measured using an automated 96-well Varioskan Ascent™ plate reader (Thermo Electron) and the presence of nitrite was quantified by comparison with an NaNO₂ standard curve. Its measured IC₅₀ was of 25±1 uM.

Although the present invention has been described hereinabove by way of specific embodiments thereof, it can be modified, without departing from the spirit and nature of the subject invention as defined in the appended claims.

1.-13. (canceled)

14. A method of administering a compound of formula (I) 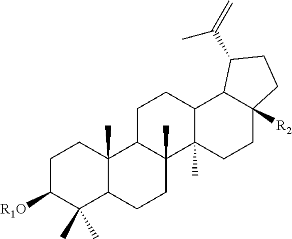
wherein
R₁ is selected from the group consisting of hydrogen, acetate, α-L-Rhamnopyranose, α-D-Mannopyranose, 13-D-Xylopyranose, 13-D-Glucopyranose, and α-D-Arabinopyranose;
R₂ is selected from CH₃, COOH, CH₂OH and COOCH₃; to a subject suffering from a cancer selected from the group consisting of melanoma, colorectal adenocarcinoma, lung carcinoma, liver carcinoma, breast adenocarcinoma, ovarian teratocarcinoma, prostate adenocarcinoma and glioma,
with the proviso that the compound of formula (I) is not a compound of formula (I)
wherein R₁ is hydrogen and R₂ is CH₃;
wherein R₁ is hydrogen and R₂ is CH₂OH;
wherein R₁ is hydrogen and R₂ is COOH;
wherein R₁ is acetate and R₂ is CH₂OH;
wherein R₁ is hydrogen and R₂ is COOCH₃;
wherein R₁ is α-L-Rhamnopyranose and R₂ is CH₃;
wherein R₁ is β-D-Glucopyranose and R₂ is CH₂OH;
wherein R₁ is β-D-Xylopyranose and R₂ is CH₂OH;
wherein R₁ is α-L-Rhamnopyranose and R₂ is COOCH₃; or
wherein R₁ is β-D-Glucopyranose and R₂ is COOH.

15. The method of claim 14, wherein R₁ is acetate and R₂ is COOH.

16. The method of claim 14, wherein R₁ is β-D-Glucopyranose and R₂ is CH₃.

17. The method of claim 14, wherein R₁ is α-D-Arabinopyranose and R₂ is CH₃.

18. The method of claim 14, wherein R₁ is α-L-Rhamnopyranose and R₂ is CH₂OH.

19. The method of claim 14, wherein R₁ is α-D-Arabinopyranose and R₂ is CH₂OH.

20. The method of claim 14, wherein R₁ is α-D-Mannopyranose and R₂ is CH₂OH.

21. The method of claim 14, wherein R₁ is 3-D-Glucopyranose and R₂ is COOCH₃.

22. The method of claim 14, wherein R₁ is α-D-Arabinopyranose and R₂ is COOCH₃.

23. The method of claim 14, wherein R₁ is α-L-Rhamnopyranose and R₂ is COOH.

24. The method of claim 14, wherein R₁ is α-D-Arabinopyranose and R₂ is COOH.

25. The method of claim 14, wherein R₁ is α-D-Mannopyranose and R₂ is COOH.

26. The method of claim 14, wherein R₁ is β-D-Xylopyranose and R₂ is COOH.

27. A method of administering methyl betulinate to a subject suffering from colorectal adenocarcinoma or lung carcinoma.

28. A method of administering 3-β-D-glucopyranose betulinic acid to a subject suffering from colorectal adenocarcinoma or lung carcinoma.

29. The method of claim 14, wherein the administration is parenteral or systemic.

30. The method of claim 14, wherein the administration is at a tumour site.

31. The method of claim 23, wherein the cancer is lung carcinoma.

32. The method of claim 31, wherein the administration is in a dosage of about 0.5 mg/kg to about 50 mg/kg.

33. The method of claim 31, wherein the administration is in a dosage of about 4 mg/kg to about 40 mg/kg.

34.-39. (canceled)

40. A method of identifying a tumor amenable to treatment with the compound of claim 1, comprising contacting a sample of cells isolated from said tumor with the compound, wherein an IC₅₀ of the compound against the sample of cells that is smaller than or equal to 50 μM in is indicative that the tumor is amenable to treatment with said compound.

41. The method of claim 40, wherein said sample of cells is from a biopsy sample from a subject.

42. The method of claim 40, wherein said sample of cells is from a biological fluid obtained from a subject.
